(19) United States
(12) Patent Application Publication
(10) Pub. No.: US 2007/0248965 A1
(43) Pub. Date: Oct. 25, 2007

(54) Compositions and methods for the detection of a nucleic acid using circular probes in a cleavage reaction

(76) Inventors: Carsten-Peter Carstens, San Diego, CA (US); Joseph A. Sorge, Wilson, WY (US)

Correspondence Address: AGILENT TECHOLOGIES INC, P.O BOX 7599, BLDG 3 , LEGAL, LOVELAND, CO, 80537-0599, US

(21) Appl. No.: 11/603,569

(22) Filed: Nov. 22, 2006

Related U.S. Application Data
(60) Provisional application No. 60/739,489, filed on Nov. 23, 2005.
(60) Provisional application No. 60/748,272, filed on Dec. 7, 2005.

(51) Int. Cl. C12Q 1/68

(52) U.S. Cl. 435/6

(57) ABSTRACT

The invention provides a method of generating a signal indicative of the presence of a target nucleic acid sequence in a sample by forming a cleavage structure by incubating a sample comprising a target nucleic acid sequence with a probe, nucleic acid polymerase, nuclease and optionally a primer. The target nucleic acid is extended and circularized and a cleavage structure is formed. The cleavage structure is cleaved with a nuclease to generate a nucleic acid fragment which is indicative of the presence of a target nucleic acid sequence in the sample.

### RELATED APPLICATIONS

This application claims the benefit of U.S. Provisional Application No. 60/739,489, filed on Nov. 23, 2005 and U.S. Provisional Application No. 60/748,272 filed on Dec. 7, 2005. The entire teachings of the above applications are incorporated herein by reference.

### BACKGROUND OF THE INVENTION

The fidelity of DNA replication, recombination, and repair is essential for maintaining genome stability, and all of these processes depend on 5′ to 3′ exonuclease enzymes which are present in all organisms. For DNA repair, these enzymes are required for damaged fragment excision and recombinational mismatch correction. For replication, these nucleases are critical for the efficient processing of Okazaki fragments during lagging strand DNA synthesis. In Escherichia coli, this latter activity is provided by DNA polymerase I (PolI); E. coli strains with inactivating mutations in the PolI 5′ to 3′ exonuclease domain are not viable due to an inability to process Okazaki fragments. Eukaryotic DNA polymerases, however, lack an intrinsic 5′□3′ exonuclease domain, and this critical activity is provided by the multifunctional, structure-specific metallonuclease FEN-1 (five′ exonuclease-1 or flap endonuclease-1), which also acts as an endonuclease for 5′ DNA flaps (Reviewed in Hosfield et al., 1998a, Cell, 95:135).

Methods of detecting and/or measuring a nucleic acid wherein an enzyme produces a labeled nucleic acid fragment are known in the art.

U.S. Pat. Nos. 5,843,669, 5,719,028, 5,837,450, 5,846,717 and 5,888,780 disclose a method of cleaving a target DNA molecule by incubating a 5′ labeled target DNA with a DNA polymerase isolated from Thermus aquaticus (Taq polymerase) and a partially complementary oligonucleotide capable of hybridizing to sequences at the desired point of cleavage. The partially complementary oligonucleotide directs the Taq polymerase to the target DNA through formation of a substrate structure containing a duplex with a 3′ extension opposite the desired site of cleavage wherein the non-complementary region of the oligonucleotide provides a 3′ arm and the unannealed 5′ region of the substrate molecule provides a 5′ arm. The partially complementary oligonucleotide includes a 3′ nucleotide extension capable of forming a short hairpin. The release of labeled fragment is detected following cleavage by Taq polymerase.

U.S. Pat. Nos. 5,843,669, 5,719,028, 5,837,450, 5,846,717 and 5,888,780 disclose the generation of mutant, thermostable DNA polymerases that have very little or no detectable synthetic activity, and wild type thermostable nuclease activity. The mutant polymerases are said to be useful because they lack 5′ to 3′ synthetic activity; thus synthetic activity is an undesirable side reaction in combination with a DNA cleavage step in a detection assay.

U.S. Pat. Nos. 5,843,669, 5,719,028, 5,837,450, 5,846,717 and 5,888,780 disclose that wild type Taq polymerase or mutant Taq polymerases that lack synthetic activity can release a labeled fragment by cleaving a 5′ end labeled hairpin structure formed by heat denaturation followed by cooling, in the presence of a primer that binds to the 3′ arm of the hairpin structure. Further, U.S. Pat. Nos. 5,843,669, 5,719,028, 5,837,450, 5,846,717 and 5,888,780 teach that the mutant Taq polymerases lacking synthetic activity can also cleave this hairpin structure in the absence of a primer that binds to the 3′ arm of the hairpin structure.

U.S. Pat. Nos. 5,843,669, 5,719,028, 5,837,450, 5,846,717 and 5,888,780 also disclose that cleavage of this hairpin structure in the presence of a primer that binds to the 3′ arm of the hairpin structure by mutant Taq polymerases lacking synthetic activity yields a single species of labeled cleaved product, while wild type Taq polymerase produces multiple cleavage products and converts the hairpin structure to a double stranded form in the presence of dNTPs, due to the high level of synthetic activity of the wild type Taq enzyme.

U.S. Pat. Nos. 5,843,669, 5,719,028, 5,837,450, 5,846,717 and 5,888,780 also disclose that mutant Taq polymerases exhibiting reduced synthetic activity, but not wild type Taq polymerase, can release a single labeled fragment by cleaving a linear nucleic acid substrate comprising a 5′ end labeled target nucleic acid and a complementary oligonucleotide wherein the complementary oligonucleotide hybridizes to a portion of the target nucleic acid such that 5′ and 3′ regions of the target nucleic acid are not annealed to the oligonucleotide and remain single stranded.

U.S. Pat. Nos. 5,843,669, 5,719,028, 5,837,450, 5,846,717 and 5,888,780 also disclose a method of cleaving a labeled nucleic acid substrate at naturally occurring areas of secondary structure. According to this method, biotin labeled DNA substrates are prepared by PCR, mixed with wild type Taq polymerase or CleavaseBN (a mutant Taq polymerase with reduced synthetic activity and wild type 5′ to 3′ nuclease activity), incubated at 950 C for 5 seconds to denature the substrate and then quickly cooled to 650 C to allow the DNA to assume its unique secondary structure by allowing the formation of intra-strand hydrogen bonds between the complementary bases. The reaction mixture is incubated at 650 C to allow cleavage to occur and biotinylated cleavage products are detected.

U.S. Pat. No. 5,843,669 discloses a method of detecting polymorphisms by cleavase fragment length polymorphism analysis using a thermostable FEN-1 nuclease in the presence or absence of a mutant Taq polymerase exhibiting reduced synthetic activity. According to this method, double stranded Hepatitis C virus (HCV) DNA fragments are labeled by using 5′ end labeled primers (labeled with TMR fluorescent dye) in a PCR reaction. The TMR labeled PCR products are denatured by heating to 95° C. and cooled to 55° C. to generate a cleavage structure. U.S. Pat. No. 5,843,669 discloses that a cleavage structure comprises a region of a single stranded nucleic acid substrate containing secondary structure. Cleavage is carried out in the presence of CleavaseBN nuclease, FEN-1 nuclease derived from the archaebacteria Methanococcus jannaschii or both enzymes. Labeled reaction products are visualized by gel electrophoresis followed by fluoroimaging. U.S. Pat. No. 5,843,669 discloses that CleavaseBN nuclease and Methanococcus jannaschii FEN-1 nuclease produce cleavage patterns that are easily distinguished from each other, and that the cleavage patterns from a reaction containing both enzymes include elements of the patterns produced by cleavage with each individual enzyme but are not merely a composite of the cleavage patterns produced by each individual enzyme. This indicates that some of the fragments that are not cleaved by one enzyme (and which appear as a band in that enzyme's pattern) can be cleaved by a second enzyme in the same reaction mixture.

Lyamichev et al. disclose a method for detecting DNAs wherein overlapping pairs of oligonucleotide probes that are partially complementary to a region of target DNA are mixed with the target DNA to form a 5′ flap region, and wherein cleavage of the labeled downstream probe by a thermostable FEN-1 nuclease produces a labeled cleavage product. Lyamichev et al. also disclose reaction conditions wherein multiple copies of the downstream oligonucleotide probe can be cleaved for a single target sequence in the absence of temperature cycling, so as to amplify the cleavage signal and allow quantitative detection of target DNA at sub-attomole levels (Lyamichev et al., 1999, Nat. Biotechnol., 17:292).

The polymerase chain reaction (PCR) technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment whose termini are defined by the 5′ ends of the primers. PCR is reported to be capable of producing a selective enrichment of a specific DNA sequence by a factor of 109. The PCR method is also described in Saiki et al., 1985, Science, 230:1350.

While the PCR technique is an extremely powerful method for amplifying nucleic acid sequences, the detection of the amplified material requires additional manipulation and subsequent handling of the PCR products to determine whether the target DNA is present. It is desirable to decrease the number of subsequent handling steps currently required for the detection of amplified material. An assay system, wherein a signal is generated while the target sequence is amplified, requires fewer handling steps for the detection of amplified material, as compared to a PCR method that does not generate a signal during the amplification step.

U.S. Pat. Nos. 5,210,015 and 5,487,972 disclose a PCR based assay for releasing labeled probe comprising generating a signal during the amplification step of a PCR reaction in the presence of a nucleic acid to be amplified, Taq polymerase that has 5′ to 3′ exonuclease activity and a 5′, 3′ or 5′ and 3′ end-labeled probe comprising a region complementary to the amplified region and an additional non-complementary 5′ tail region. U.S. Pat. Nos. 5,210,015 and 5,487,972 disclose further that this PCR based assay can liberate the 5′ labeled end of a hybridized probe when the Taq polymerase is positioned near the labeled probe by an upstream probe in a polymerization independent manner, e.g. in the absence of dNTPs.

### SUMMARY OF THE INVENTION

The invention provides a method of generating a signal indicative of the presence of a target nucleic acid sequence in a sample by forming a cleavage structure by incubating a sample comprising a target nucleic acid sequence with a probe, nucleic acid polymerase, nuclease and optionally a primer. The target nucleic acid is extended and circularized and a cleavage structure is formed. The cleavage structure is cleaved with a nuclease to generate a nucleic acid fragment which is indicative of the presence of a target nucleic acid sequence in the sample.

In one aspect, the present invention provides a method of generating a signal indicative of the presence of a target nucleic acid in a sample, wherein the signal generated indicates the presence of an extension sequence resulting from extension of the target nucleic acid, the method comprising:
a) extending the target nucleic acid to add an additional region;
b) circularizing the extended target nucleic acid;
c) forming a cleavage structure by incubating the sample with a nucleic acid polymerase, the cleavage structure comprising duplex and single-stranded nucleic acid, wherein the single-stranded nucleic acid comprises a 5′ flap;
d) cleaving the cleavage structure with a nuclease to release a nucleic acid fragment from the cleavage structure; and
e) detecting and/or measuring the release of the nucleic acid fragment as an indication of the presence of the target nucleic acid.

In another aspect, the present invention provides a method of generating a signal indicative of the presence of a target nucleic acid in a sample, wherein the signal generated indicates the presence of an extension sequence resulting from extension of the target nucleic acid, the method comprising:
a) extending the target nucleic acid to add an additional region;
b) circularizing the extended target nucleic acid;
c) forming a cleavage structure by incubating the sample with a primer which hybridizes to the circularized and extended target nucleic acid and extending the 3′ end of the primer with a nucleic acid polymerase and displacing the 5′ end of the primer;
d) cleaving the cleavage structure with a nuclease to release a nucleic acid fragment from the cleavage structure; and
e) detecting and/or measuring the release of the nucleic acid fragment as an indication of the presence of the target nucleic acid.

In still another aspect, the present invention provides a method for forming a cleavage structure in a sample, cleaving the cleavage structure to produce a cleavage product that is indicative of the presence of the target nucleic acid, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region; and
      B) an upstream region; and
   ii) the nucleic acid probe comprises:
      A) a 5′ region complementary to the upstream region on the target nucleic acid;
      B) a 3′ region complementary to the 3′ region on the target nucleic acid; and
      C) an additional region;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region downstream of the 3′ region and complementary to the additional region of the nucleic acid probe, followed by circularizing the extended target nucleic acid;
d) removing the nucleic acid probe from the extended target nucleic acid;
e) annealing a primer to the extended target nucleic acid, wherein the primer has a sequence that is complementary to at least a portion of the primer binding region on the extended target nucleic acid and primes the synthesis of a complementary nucleic acid strand;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In yet another aspect, the present invention provides a method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region (Y2); and
      B) a region (Y1) upstream to Y2; and
   ii) the nucleic acid probe comprises:
      A) a 5′ region (Y1′) complementary to Y1;
      B) a 3′ region (Y2′) complementary to Y2 and
      C) an additional binding region (P′) between Y1′ and Y2′;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region (P), wherein P is downstream of Y2 and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid and primes the synthesis of a complementary nucleic acid strand;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In still another aspect, the present invention provides a method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region (B);
      B) a region (Y2) upstream to B; and
      C) a region (Y1) upstream to Y2; and
   ii) the nucleic acid probe comprises:
      A) a 5′ region (Y1′) complementary to Y1;
      B) a 3′ region (Y2′) complementary to Y2;
      C) a region (B′) upstream to Y2′; and
      D) an additional region (P′) between Y1′and B′;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region (P), wherein P is downstream of B and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid upstream of a sequence complementary to at least a portion of region Y2 and primes the synthesis of a complementary nucleic acid strand while looping out region B of the extended target nucleic acid;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
e) detecting and/or measuring the release of the nucleic acid fragment as an indication of the presence of the target nucleic acid.

In still another aspect, the present invention provides a method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region (Y);
      B) a region (X) upstream of Y; and
      C) a series of intervening regions (A, Z1, Z2, B) between X and Y in the order of X-A-Z1-Z2-B-Y;
   ii) the nucleic acid probe comprises:
      A) a 5′ region (Z1′) complementary to Z1;
      B) a 3′ region (Z2′) complementary to Z2;
      C) a region (X′) complementary to X;
      D) a region (Y′) complementary to Y; and
      E) an additional region (P′) between X1′ and Y1′;
b) annealing the nucleic acid probe to the target nucleic acid and priming synthesis of an extended target nucleic acid while looping out one or more portions of regions A and B of the target sequence;
c) incubating the sample with a nucleic acid polymerase and extending the 3′ end of the target nucleic acid to generate an extended target nucleic acid having a primer binding region (P), wherein P is downstream of Y and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid upstream of a sequence complementary to at least a portion of region Z2 and primes the synthesis of a complementary nucleic acid strand while looping out one or more portions of regions Y and B of the extended target nucleic acid;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In another aspect, the present invention provides a kit for generating a signal indicative of the presence of a target nucleic acid sequence of a target nucleic acid in a sample, comprising a nucleic acid polymerase, a FEN nuclease, a suitable buffer, a nucleic acid probe, wherein:
i) the target nucleic acid comprises:
   A) a 3′ region; and
   B) an upstream region; and
ii) the nucleic acid probe comprises:
   A) a 5′ region complementary to the upstream region on the target nucleic acid;
   B) a 3′ region complementary to the 3′ region on the target nucleic acid; and
   C) an additional region.

As used herein a “nuclease” or a “cleavage agent” refers to an enzyme that is specific for, that is, cleaves a cleavage structure according to the invention and is not specific for, that is, does not substantially cleave either a probe or a primer that is not hybridized to a target nucleic acid, or a target nucleic acid that is not hybridized to a probe or a primer. The term “nuclease” includes an enzyme that possesses 5′ endonucleolytic activity for example a DNA polymerase, e.g. DNA polymerase I from E. coli, and DNA polymerase from Thermus aquaticus (Taq), Thermus thermophilus (Tth), and Thermus flavus (Tfl). A nuclease according to the invention also includes Saccharomyces cerevisiae RAD27, and Schizosaccharomyces pombe RAD2, Pol I DNA polymerase associated 5′ to 3′ exonuclease domain, (e.g. E. coli, Thermus aquaticus (Taq), Thermus flavus (Tfl), Bacillus caldotenax (Bca), Streptococcus pneumoniae) and phage functional homologs of FEN including but not limited to T5 5′ to 3′ exonuclease, T7 gene 6 exonuclease and T3 gene 6 exonuclease. Preferably, only the 5′ to 3′ exonuclease domains of Taq, Tfl and Bca nuclease are used. The term “nuclease” does not include RNAse H.

As used herein a “FEN nuclease” refers to an enzyme that cleaves a cleavage structure according to the invention. The term “FEN nuclease” encompasses an enzyme that consists essentially of a 5′ exonuclease and/or an endonuclease activity. As used herein, “consists essentially of” refers to an enzyme wherein the predominant activity of the enzyme is a 5′ exonucleolytic and/or endonucleolytic activity, such that one or both of 5′ to 3′ synthetic activity and 3′ single-stranded flap cleavage activity (i.e., 3′ endonucleolytic and/or 3′ exonucleolytic activity) are substantially lacking. “Substantially lacks” means that the FEN nuclease possesses no more than 5% or 10% and preferably less than 0.1%, 0.5%, or 1% of the activity of a wild type enzyme (e.g. for 5′ to 3′ synthetic activity and the 3′ endonucleolytic and/or ′3 exonucleolytic activities, the enzyme may be a wild type DNA polymerase having these activities). 5′ to 3′ synthetic activity can be measured, for example, in a nick translation assay or an enzymatic sequencing reaction which involve the formation of a phosphodiester bridge between the 3′-hydroxyl group at the growing end of an oligonucleotide primer and the 5′-phosphate group of an incoming deoxynucleotide, such that the overall direction of synthesis is in the 5′ to 3′ direction. 3′ flap cleavage may be measured in a DNA synthesis reaction in which, because the (labelled) 3′ end of a DNA duplex is unpaired, it is cleaved from the duplex. A FEN nuclease that “consists of” a 5′ exonuclease and/or endonuclease activity refers to an enzyme that “lacks” 5′ to 3′ synthetic activity and/or 3′ single-stranded flap cleavage activity. “Lacks” means that the Fen nuclease has no detectable activity or has only “minor” activity, i.e., less than 1.0%, 0.5%, 0.1% or 0.01% of the activity of a wild type enzyme. As used herein, “FEN nuclease” encompasses a 5′ flap-specific nuclease.

The term “FEN nuclease” also embodies a 5′ flap-specific nuclease. A nuclease or cleavage agent according to the invention includes but is not limited to a FEN nuclease enzyme derived from Archaeglobus fulgidus, Methanococcus jannaschii, Pyrococcus furiosus, human, mouse or Xenopus laevis.

As used herein, “wild type” refers to a gene or gene product which has the characteristics of (i.e., either has the sequence of or encodes, for the gene, or possesses the sequence or activity of, for an enzyme) that gene or gene product when isolated from a naturally occurring source.

A “5′ flap-specific nuclease” (also referred to herein as a “flap-specific nuclease”) according to the invention is an endonuclease which can remove a single stranded flap that protrudes as a 5′ single strand. A flap-specific nuclease according to the invention can also cleave a pseudo-Y structure. A substrate of a flap-specific nuclease according to the invention, comprises a target nucleic acid, a second nucleic acid, a portion of which specifically hybridizes with a target nucleic acid, and a primer extension product from a third nucleic acid that specifically hybridizes with a target nucleic acid sequence.

As used herein, a “cleavage structure” refers to a polynucleotide structure comprising at least a duplex nucleic acid having a single stranded region comprising a flap, a loop, a single-stranded bubble, a D-loop, a nick or a gap. A cleavage structure according to the invention thus includes a polynucleotide structure comprising a flap strand of a branched DNA wherein a 5′ single-stranded polynucleotide flap extends from a position near its junction to the double stranded portion of the structure. In some embodiments, the flap is labelled with a detectable label. A flap of a cleavage structure according to the invention is preferably about 1-10,000 nucleotides and more preferably about 2-25 nucleotides and is preferably cleaved at a position located at the phosphate positioned at the “elbow” of the branched structure or at any of one to ten phosphates located proximal and/or distal from the elbow of the flap strand. As used herein, “elbow” refers to the phosphate bond between the first single stranded nucleotide of the 5′ flap and the first double stranded (e.g., hybridized to the target nucleic acid) nucleotide. In one embodiment, the flap is created by the displacement of the 5′ end of an oligonucleotide by an invading nucleic acid strand.

A cleavage structure according to the invention can comprise a target nucleic acid sequence with a 5′ flap, and a probe that specifically hybridizes with a portion of the target nucleic acid. For example, a cleavage structure according to the invention may comprise a target nucleic acid, a circularized 3′ polymerase extended portion of the target sequence, and a probe that is complementary to the target sequence and the 3′ polymerase extended portion of the target. (For example, see FIG. 1).

In another embodiment, a cleavage structure according to the invention may comprise a circularized target nucleic acid, an upstream oligonucleotide, e.g., extended 3′ portion of the primer, and a downstream oligonucleotide, e.g., 5′ portion of the primer. (For example, see FIG. 2).

A cleavage structure according to the invention may be formed by hybridizing a target nucleic acid sequence with an oligonucleotide wherein the oligonucleotide comprises a complementary region that anneals to the target nucleic acid sequence, and a non-complementary region that does not anneal to the target nucleic acid sequence and forms a 5′ flap.

A cleavage structure also may be a pseudo-Y structure wherein a pseudo Y-structure is formed if the strand upstream of a flap (referred to herein as a flap adjacent strand or primer strand) is removed, and double stranded DNA substrates containing a gap or nick. A “cleavage structure”, as used herein, does not include a double stranded nucleic acid structure with only a 3′ single-stranded flap. As used herein, a “cleavage structure” comprises ribonucleotides or deoxyribonucleotides and thus can be RNA or DNA.

A cleavage structure according to the invention may be an overlapping flap wherein the 3′ end of an upstream oligonucleotide, e.g., extended 3′ portion of a primer, capable of hybridizing to a circularized target nucleic acid sequence is complementary to 1 base pair of the downstream oligonucleotide, e.g., 5′ portion of the primer, that is annealed to the circularized target nucleic acid sequence and wherein the overlap is directly downstream of the point of extension of the single stranded flap.

A cleavage structure according to the invention may be a polynucleotide structure comprising a flap extending from the oligonucleotide, wherein the flap is formed by extension of the 3′ portion of the oligonucleotide by the synthetic activity of a nucleic acid polymerase, and subsequent, partial, displacement of its 5′ end.

A cleavage structure according to the invention is formed by the steps of 1: incubating a) an extendable 3′ end of a primer (P″ of FIG. 3) and b) a extended circular target (Y1, Y2, P′ of FIG. 3) c) a suitable buffer, under conditions that allow the primer to hybridize to the extended circular target, and 2: extending the 3′ end of the primer oligonucleotide by the synthetic activity of a polymerase such that the newly synthesized 3′ end of the primer oligonucleotide becomes adjacent to and/or displaces at least a portion of (i.e., at least 1-10 nucleotides of) the 5′ end of the primer. According to the method of the invention, buffers and extension temperatures are favorable for strand displacement by a particular nucleic acid polymerase according to the invention.

In a preferred embodiment of the invention a cleavage structure is labelled.

As used herein, “label” or “labelled moiety capable of providing a signal” refers to any atom or molecule which can be used to provide a detectable (preferably quantifiable) signal, and which can be operatively linked to a nucleic acid. Labels may provide signals detectable by fluorescence, radioactivity, colorimetry, gravimetry, X-ray diffraction or absorption, magnetism, enzymatic activity, mass spectrometry, binding affinity, hybridization radiofrequency and the like.

As used herein, “generating a signal” refers to detecting and or measuring a released nucleic acid fragment as an indication of the presence of a target nucleic acid sequence in a sample.

As used herein, “sample” refers to any substance containing or presumed to contain a nucleic acid of interest (a target nucleic acid sequence) or which is itself a nucleic acid containing or presumed to contain a target nucleic acid sequence of interest. The term “sample” thus includes a sample of nucleic acid (genomic DNA, cDNA, RNA), cell, organism, tissue, fluid, or substance including but not limited to, for example, plasma, serum, spinal fluid, lymph fluid, synovial fluid, urine, tears, stool, external secretions of the skin, respiratory, intestinal and genitourinary tracts, saliva, blood cells, tumors, organs, tissue, samples of in vitro cell culture constituents, natural isolates (such as drinking water, seawater, solid materials), microbial specimens, and objects or specimens that have been “marked” with nucleic acid tracer molecules.

As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template, and if possessing a 5′ to 3′ nuclease activity, hydrolyzing intervening, annealed probe to release both labeled and unlabeled probe fragments, until synthesis terminates. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

As used herein, “5′ to 3′ exonuclease activity” or “5′ to 3′ exonuclease activity” refers to that activity of a template-specific nucleic acid polymerase e.g. a 5′ to 3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow (Klenow et al., 1970, Proc. Natl. Acad. Sci., USA, 65:168) fragment does not, (Klenow et al., 1971, Eur. J. Biochem., 22:371)), or polynucleotides are removed from the 5′ end by an endonucleolytic activity that may be inherently present in a 5′ to 3′ exonuclease activity.

As used herein, the phrase “substantially lacks 5′ to 3′ exonuclease activity” or “substantially lacks 5′ to 3′ exonuclease activity” means having less than 10%, 5%, 1%, 0.5%, or 0.1% of the activity of a wild type enzyme. The phrase “lacking 5′ to 3′ exonuclease activity” or “lacking 5′ to 3′ exonuclease activity” means having undetectable 5′ to 3′ exonuclease activity or having less than about 1%, 0.5%, or 0.1% of the 5′ to 3′ exonuclease activity of a wild type enzyme. 5′ to 3′ exonuclease activity may be measured by an exonuclease assay which includes the steps of cleaving a nicked substrate in the presence of an appropriate buffer, for example 10 mM Tris-HCl (pH 8.0), 10 mM MgCl₂ and 50 μg/ml bovine serum albumin) for 30 minutes at 60° C., terminating the cleavage reaction by the addition of 95% formamide containing 10 mM EDTA and 1 mg/ml bromophenol blue, and detecting nicked or un-nicked product.

Nucleic acid polymerases useful according to the invention include but are not limited to Pfu, exo− Pfu (a mutant form of Pfu that lacks 3′ to 5′ exonuclease activity), the Stoffel fragment of Taq, N-truncated Bst, N-truncated Bca, Genta, JdF3 exo−, Vent, Vent exo− (a mutant form of Vent that lacks 3′ to 5′ exonuclease activity), Deep Vent, Deep Vent exo− (a mutant form of Deep Vent that lacks 3′ to 5′ exonuclease activity), U1Tma and Sequenase. Additional nucleic acid polymerases useful according to the invention are included below in the section entitled, “Nucleic Acid Polymerases”.

As used herein, “cleaving” refers to enzymatically separating a cleavage structure into distinct (i.e. not physically linked to other fragments or nucleic acids by phosphodiester bonds) fragments or nucleotides and fragments that are released from the cleavage structure. For example, cleaving a labelled cleavage structure refers to separating a labelled cleavage structure according to the invention and defined below, into distinct fragments including fragments derived from an oligonucleotide that specifically hybridizes with a target nucleic acid sequence or wherein one of the distinct fragments is a labelled nucleic acid fragment derived from a target nucleic acid sequence and/or derived from an oligonucleotide that specifically hybridizes with a target nucleic acid sequence that can be detected and/or measured by methods well known in the art and described herein that are suitable for detecting the labelled moiety that is present on a labelled fragment.

As used herein, “endonuclease” refers to an enzyme that cleaves bonds, preferably phosphodiester bonds, within a nucleic acid molecule. An endonuclease according to the invention can be specific for single-stranded or double-stranded DNA or RNA.

As used herein, “exonuclease” refers to an enzyme that cleaves bonds, preferably phosphodiester bonds, between nucleotides one at a time from the end of a polynucleotide. An exonuclease according to the invention can be specific for the 5′ or 3′ end of a DNA or RNA molecule, and is referred to herein as a 5′ exonuclease or a 3′ exonuclease.

As used herein a “flap” refers to a region of single stranded DNA that extends from a double stranded nucleic acid molecule. A flap according to the invention is preferably between about 1-10,000 nucleotides, more preferably between about 2-25 nucleotides and most preferably between about 5-20 nucleotides.

As used herein, “detecting a target nucleic acid sequence” or “measuring a target nucleic acid sequence” refers to determining the presence of a particular target nucleic acid sequence in a sample or determining the amount of a particular target nucleic acid sequence in a sample as an indication of the presence of a target nucleic acid sequence in a sample. The amount of a target nucleic acid sequence that can be measured or detected is preferably about 1 molecule to 10²⁰ molecules, more preferably about 100 molecules to 10¹⁷ molecules and most preferably about 1000 molecules to 10¹⁴ molecules. According to one embodiment of the invention, the detected nucleic acid is derived from the labelled 5′ end of a target nucleic acid or primer of a cleavage structure according to the invention, that is displaced after extension by a polymerase of the target nucleic acid or primers 3′ end. According to the present invention, a label is attached to the 5′ end of the target or primer. Alternatively, the primer has a label and a quencher which are separated by a cleavage site. When the nuclease cleaves the cleavage site the label and quencher are separated thus generating a detectable signal.

According to the invention, the oligonucleotides may be labelled internally. As used herein, “detecting the release of nucleic acid fragments” or “measuring the release of nucleic acid fragments” refers to determining the presence of a nucleic acid in a sample or determining the amount of a nucleic acid fragment in a sample. Methods well known in the art and described herein can be used to detect or measure release of nucleic acid fragments. The nucleic acid fragments can be detected directly, e.g., fluorescent signal from a FRET pair, or indirectly, e.g., secondary cleavage or amplification reaction. (See other related patents, the disclosures of which are incorporated herein by reference for direct detection (U.S. patent application Ser. No. 10/981,942, filed Nov. 5, 2004; U.S. Pat. No. 6,528,254 B1, filed Oct. 29, 1999; U.S. Pat. No. 6,548,250, filed Aug. 30, 2000) and indirect detection (U.S. Pat. No. 6,893,819, filed Nov. 21, 2000; U.S. Application 60/725,916, filed Oct. 11, 2005.)

As used herein, “nucleic acid fragments” refer to cleaved mononucleotides or small oligonucleotides or oligonucleotides derived from the cleavage structure according to the invention wherein the cleaved oligonucleotides are preferably between about 2-1000 nucleotides, more preferably between about 2-50 nucleotides and most preferably between about 5-20 nucleotides, which are cleaved from a cleavage structure by a nuclease and can be detected by methods well known in the art and described herein.

As used herein, “thermostable” refers to an enzyme which is stable and active at temperatures as great as preferably between about 90-100° C. and more preferably between about 70-98° C. to heat as compared, for example, to a non-thermostable form of an enzyme with a similar activity. For example, a thermostable nucleic acid polymerase or FEN nuclease derived from thermophilic organisms such as P. furiosus, M. jannaschii, A. fulgidus or P. horikoshii are more stable and active at elevated temperatures as compared to a nucleic acid polymerase from E. coli or a mammalian FEN enzyme. A representative thermostable nucleic acid polymerase isolated from Thermus aquaticus (Taq) is described in U.S. Pat. No. 4,889,818 and a method for using it in conventional PCR is described in Saiki et al., 1988, Science 239:487. Another representative thermostable nucleic acid polymerase isolated from P. furiosus (Pfu) is described in Lundberg et al., 1991, Gene, 108:1-6. Additional representative temperature stable polymerases include, e.g., polymerases extracted from the thermophilic bacteria Thermus flavus, Thermus ruber, Thermus thermophilus, Bacillus stearothermophilus (which has a somewhat lower temperature optimum than the others listed), Thermus lacteus, Thermus rubens, Thermotoga maritima, or from thermophilic archaea Thermococcus litoralis, and Methanothermus fervidus.

Temperature stable polymerases and FEN nucleases are preferred in a thermocycling process wherein double stranded nucleic acids are denatured by exposure to a high temperature (about 95° C.) during the PCR cycle.

In another preferred embodiment, the FEN nuclease is a flap-specific nuclease.

In another preferred embodiment, the FEN nuclease is thermostable.

In another preferred embodiment, the cleavage structure is formed comprising at least one labeled moiety capable of providing a signal.

In another preferred embodiment, the cleavage structure is formed comprising a pair of interactive signal generating labeled moieties effectively positioned to quench the generation of a detectable signal, wherein the labeled moieties are separated by a site susceptible to FEN nuclease cleavage, thereby allowing the nuclease activity of the FEN nuclease to separate the first interactive signal generating labeled moiety from the second interactive signal generating labeled moiety by cleaving at the site susceptible to FEN nuclease, thereby generating a detectable signal.

As used herein, an “oligonucleotide primer” or “extension primer” refers to a single stranded DNA or RNA molecule that can hybridize to a nucleic acid template and primes enzymatic synthesis of a second nucleic acid strand. Oligonucleotide primers useful according to the invention are between about 10 to 100 nucleotides in length, preferably about 17-50 nucleotides in length and more preferably about 17-45 nucleotides in length.

As used herein, “target nucleic acid sequence” or “template nucleic acid sequence” refers to a region of a nucleic acid that is to be either replicated, amplified, and/or detected. In one embodiment, the “target nucleic acid sequence” or “template nucleic acid sequence” resides between two primer sequences used for amplification. A “target nucleic acid” can comprise deoxyribonucleic acid, ribonucleic acid or mixtures thereof. As used herein, a “target nucleic acid” has a defined 5′ end and 3′ end. In addition, a “target nucleic acid” can further comprise non-natural nucleic acids. Generally, a “target nucleic acid” is of a sufficient length to hybridize to two at least target nucleic acid binding sites of the probe (Y1′ and Y2′ of FIG. 3), and is therefore generally at least 10 bases in length, typically at least 30 bases in length, for example, at least 35, 40, or 45 bases in length. While the target nucleic acid can be large nucleic acid fragments, it is generally limited to nucleic acids of 20 kilobases or less.

As used herein, a “circularizing the extended target nucleic acid” refers to a circular nucleic acid that is formed by nucleic acid synthesis using the probe as a template from the circular hybridization complex formed between a target nucleic acid and a probe. The “circularized target nucleic acid” forms as a result of ligating the original target nucleic acid with its 3′ extended synthesis product.

As used herein, a “probe” refers to a type of oligonucleotide having or containing a sequence which is complementary to another polynucleotide, e.g., a target nucleic acid. The probe of the present invention can be of any length, so long as it is capable of forming a hybridization complex with the target nucleic acid (e.g., it must contain two target nucleic acid binding sites), and also allowing for effective extension reaction from the end of the target nucleic acid. The probe of the present invention is generally between 30 and 1 kb in length, typically between 100 and 200 bases in length.

The probe of the present invention comprises two “target nucleic acid binding sites.” As used herein, a “target nucleic acid binding site” and “TNA binding site” refer to a region within the probe which is complementary to a portion of the target nucleic acid. For example, a “first target nucleic acid binding site” of the probe is complementary to and hybridizes to a “first probe interacting site” of the target nucleic acid. A “target nucleic acid binding site” is generally between 10 and 40 bases in length, typically between 15 and 25 bases in length.

A target nucleic acid binding site can be located at the “5′ end” or “3′ end” of the probe. As used herein, a target nucleic acid binding site, the most proximal nucleotide of which is located within 5 bases from the 5′ terminus of the probe, is said to be “at the 5′ end” of the probe. Similarly, a target nucleic acid binding site, the most proximal nucleotide of which is located within 5 bases from the 3′ terminus of the probe, is said to be “at the 3′ end” of the probe.

Generally the 3′ terminus of the probe will be “blocked” to prohibit creation of an extension product. “Blocking” can be achieved by using non-complementary bases at or near the 3′ terminus, or by adding a chemical moiety such as biotin or a phosphate group to the 3′ hydroxyl of the last nucleotide. Blocking can also be achieved by removing the 3′-OH or by using a nucleotide that lacks a 3′-OH such as dideoxynucleotide, or by other methods known to one skilled in the art.

The 5′ terminus of the probe will generally be modified such that it is “not ligatable” or “non-ligatable.” To modify the probe to be “non ligatable”, the 5′ phosphoryl moiety can be removed or replaced, or an additional moiety can be attached to the 5′ phosphoryl moiety to prevent ligation with another nucleic acid, for example, by a ligase enzyme.

### BRIEF DESCRIPTION OF THE DRAWINGS

FIG. 1 illustrates a cleavage structure of the invention utilizing a target nucleic acid, a probe, and a 3′ extension product of the target nucleic acid.

FIG. 2 is a diagram illustrating a synthesis and cleavage reaction to generate a signal according to the invention.

FIG. 3 is a schematic depicting an embodiment of the invention relating to a circular cleavage reaction using a 3′ blocked probe with an intervening sequence and a primer complementary to at least a portion of the intervening sequence.

FIG. 4 is a schematic depicting an embodiment of the invention relating to a circular cleavage reaction using a 3′ blocked probe with an intervening sequence and a primer, which is complementary to at least a portion of the intervening sequence and which displaces a portion of the target nucleic acid.

FIG. 5 is a schematic depicting an embodiment of the invention relating to a circular cleavage reaction using a 3′ blocked probe, which has an intervening sequence and which displaces one or more portions of the target nucleic acid, and a primer, which is complementary to at least a portion of the intervening sequence and which displaces a portion of the target nucleic acid.

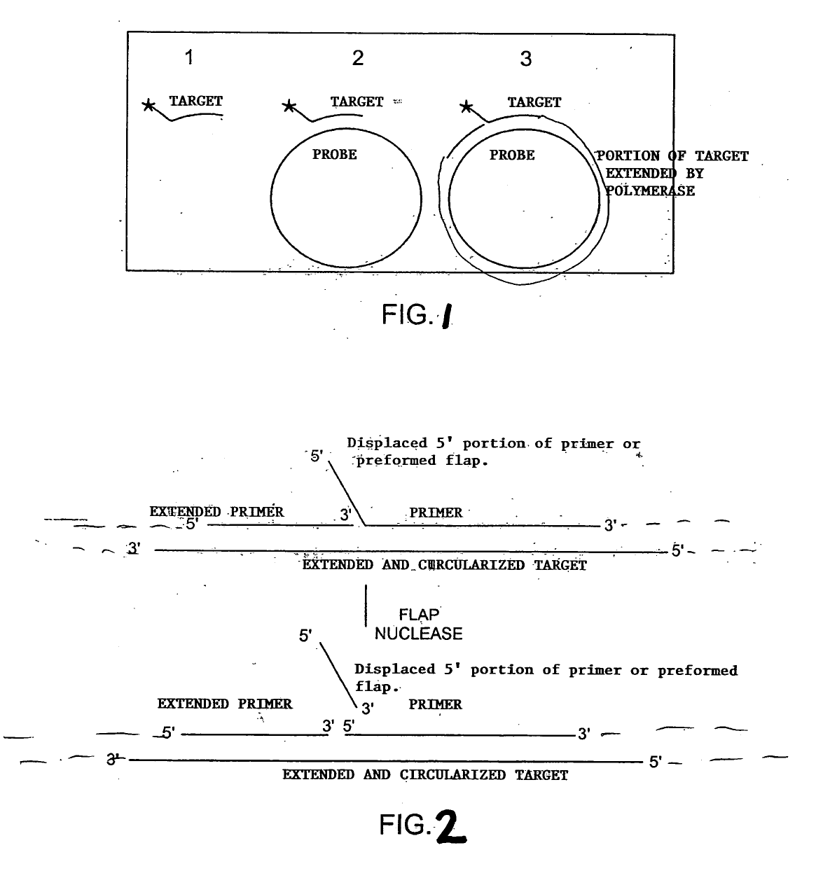

### DETAILED DESCRIPTION OF THE INVENTION

The invention provides for a method of generating a signal to detect the presence of a target nucleic acid in a sample wherein a nucleic acid is treated with the combination of a nucleic acid polymerase and a nuclease.

The invention provides a method of generating a signal indicative of the presence of a target nucleic acid in a sample, which includes the steps of extending a target nucleic acid, circularizing the extended target nucleic acid, forming a cleavage structure by incubating a extended target nucleic acid with a primer and a nucleic acid polymerase, and cleaving the cleavage structure with a nuclease to release a nucleic acid fragment and thus generate a signal. Generation of the signal is indicative of the presence of a target nucleic acid in the sample, and the signal may be detected or measured by directly detecting and/or measuring the amount of a fragment or indirectly detecting and/or measuring the amount of the fragment.

In a first aspect, the present invention provides a method of generating a signal indicative of the presence of a target nucleic acid in a sample, wherein the signal generated indicates the presence of an extension sequence resulting from extension of the target nucleic acid, the method comprising:
a) extending the target nucleic acid to add an additional region;
b) circularizing the extended target nucleic acid;
c) forming a cleavage structure by incubating the sample with a nucleic acid polymerase, the cleavage structure comprising duplex and single-stranded nucleic acid, wherein the single-stranded nucleic acid comprises a 5′ flap;
d) cleaving the cleavage structure with a nuclease to release a nucleic acid fragment from the cleavage structure; and
e) detecting and/or measuring the release of the nucleic acid fragment as an indication of the presence of the target nucleic acid.

In addition, the present invention provides a method of generating a signal indicative of the presence of a target nucleic acid in a sample, wherein the signal generated indicates the presence of an extension sequence resulting from extension of the target nucleic acid, the method comprising:
a) extending the target nucleic acid to add an additional region;
b) circularizing the extended target nucleic acid;
c) forming a cleavage structure by incubating the sample with a primer which hybridizes to the circularized and extended target nucleic acid and extending the 3′ end of the primer with a nucleic acid polymerase and displacing the 5′ end of the primer;
d) cleaving the cleavage structure with a nuclease to release a nucleic acid fragment from the cleavage structure; and
e) detecting and/or measuring the release of the nucleic acid fragment as an indication of the presence of the target nucleic acid.

In one embodiment, the nucleic acid polymerase substantially lacks 5′ to 3′ exonuclease activity. Preferably, the nucleic acid polymerase is a DNA polymerase. Preferably, the nucleic acid polymerase is thermostable. More preferably, the nucleic acid polymerase is selected from the group consisting of 5′ to 3′ exonuclease deficient Taq polymerase and Pfu polymerase.

In another embodiment, a cleavage structure is formed comprising at least one labeled moiety capable of providing a signal.

In another embodiment, the nuclease comprises a FEN nuclease. Preferably, the FEN nuclease is a flap-specific nuclease. Preferably, the FEN nuclease is thermostable. More preferably, it is selected from the group consisting of FEN nuclease enzyme derived from Archaeglobus fulgidus, Methanococcus jannaschii, and Pyrococcus furiosus. The nuclease may be derived from Taq, Tfl and Bca.

Preferably, the cleavage structure formed comprises a pair of interactive signal generating labeled moieties effectively positioned to quench the generation of a detectable signal, the labeled moieties being separated by a site susceptible to FEN nuclease cleavage, thereby allowing the nuclease activity of the FEN nuclease to separate the first interactive signal generating labeled moiety from the second interactive signal generating labeled moiety by cleaving at the site susceptible to FEN nuclease, thereby generating a detectable signal. More preferably, the pair of interactive signal generating moieties comprises a quencher moiety and a fluorescent moiety.

In another embodiment, the interactive signal generating labeled moieties are a fluorophore and a quencher. The quencher may be on the displaced 5′ region of the primer (e.g., a 5′ flap), and the fluorophore is on the complementary region of the primer (that is, the portion of the primer which forms a duplex with the circularized and extended target). Alternatively, the fluorophore is specifically attached to the second nucleotide residue of the 5′ end of the complementary region of the extended primer.

In one embodiment, the cleavage structure comprises at least one oligonucleotide primer.

In another embodiment, the method further comprises quantifying the released nucleic acid fragment to calculate the amount of target nucleic acid in the sample.

The invention also provides a method for forming a cleavage structure in a sample, cleaving the cleavage structure to produce a cleavage product that is indicative of the presence of the target nucleic acid, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region; and
      B) an upstream region; and
   ii) the nucleic acid probe comprises:
      A) a 5′ region complementary to the upstream region on the target nucleic acid;
      B) a 3′ region complementary to the 3′ region on the target nucleic acid; and
      C) an additional region;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region downstream of the 3′ region and complementary to the additional region of the nucleic acid probe, followed by circularizing the extended target nucleic acid;
d) removing the nucleic acid probe from the extended target nucleic acid;
e) annealing a primer to the extended target nucleic acid, wherein the primer has a sequence that is complementary to at least a portion of the primer binding region on the extended target nucleic acid and primes the synthesis of a complementary nucleic acid strand;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In one embodiment, the nucleic acid polymerase substantially lacks 5′ to 3′ exonuclease activity. Preferably, the nucleic acid polymerase is a DNA polymerase. Preferably, the nucleic acid polymerase is thermostable. More preferably, the nucleic acid polymerase is selected from the group consisting of 5′ to 3′ exonuclease deficient Taq polymerase and Pfu polymerase.

In another embodiment, a cleavage structure is formed comprising at least one labeled moiety capable of providing a signal.

In another embodiment, the nuclease comprises a FEN nuclease. Preferably, the FEN nuclease is a flap-specific nuclease. Preferably, the FEN nuclease is thermostable. More preferably, it is selected from the group consisting of FEN nuclease enzyme derived from Archaeglobus fulgidus, Methanococcus jannaschii, and Pyrococcus furiosus. The nuclease may be derived from Taq, Tfl and Bca.

Preferably, the cleavage structure formed comprises a pair of interactive signal generating labeled moieties effectively positioned to quench the generation of a detectable signal, the labeled moieties being separated by a site susceptible to FEN nuclease cleavage, thereby allowing the nuclease activity of the FEN nuclease to separate the first interactive signal generating labeled moiety from the second interactive signal generating labeled moiety by cleaving at the site susceptible to FEN nuclease, thereby generating a detectable signal. More preferably, the pair of interactive signal generating moieties comprises a quencher moiety and a fluorescent moiety.

In one embodiment, the cleavage structure comprises at least one oligonucleotide primer.

In another embodiment, the method further comprises quantifying the released nucleic acid fragment to calculate the amount of target nucleic acid in the sample.

In yet another embodiment, the 3′ end of the target nucleic acid in step a comprises a 3′ hydroxyl group.

In still another embodiment, the nucleic acid probe has a blocked 3′ end. Preferably, the blocked 3′ end comprises a base that is non-complementary to the target nucleic acid or a modification that inhibits addition of a nucleotide triphosphate under conditions which permit the nucleic acid synthesis or extension.

Preferably, one or both of the blocked 3′ end comprises:
a) a dideoxynucleotide;
b) a nucleotide wherein the 3′ hydroxyl has been replaced with a phosphate group; or
c) a nucleotide with a reporter moiety attached to the 3′ carbon or to the 3′ oxygen.

In yet another embodiment, the circularization of the extended target nucleic acid comprises:
i) self-ligation of the extended target nucleic acid; or
ii) cleavage of a non-complementary 5′ portion of the extended target and self-ligation.

In still another embodiment, the primer binding region P comprises about 15-20 nucleotides in length.

In another embodiment, the method further comprises quantifying the released nucleic acid fragment to calculate the amount of target nucleic acid in the sample.

In another embodiment, steps a)-g) take place simultaneously after formation of the first cleavage structure.

The invention also provides a method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region (Y2); and
      B) a region (Y1) upstream to Y2; and
   ii) the nucleic acid probe comprises:
      A) a 5′ region (Y1′) complementary to Y1;
      B) a 3′ region (Y2′) complementary to Y2; and
      C) an additional binding region (P′) between Y1′ and Y2′;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region (P), wherein P is downstream of Y2 and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid and primes the synthesis of a complementary nucleic acid strand;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In one embodiment, the nucleic acid probe has a blocked 3′ end.

In another embodiment, the nucleic acid polymerase substantially lacks 5′ to 3′ exonuclease activity.

In still another embodiment, Y1′, Y2′, P′, and P comprise about 15-20 nucleotides in length.

In a preferred embodiment, the nuclease comprises a FEN nuclease. More preferably, it is selected from the group consisting of FEN nuclease enzyme derived from Archaeglobus fulgidus, Methanococcus jannaschii, and Pyrococcus furiosus. The nuclease may be derived from Taq, Tfl and Bca.

The invention also provides a method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region (B);
      B) a region (Y2) upstream to B; and
      C) a region (Y1) upstream to Y2; and
   ii) the nucleic acid probe comprises:
      A) a 5′ region (Y1′) complementary to Y1;
      B) a 3′ region (Y2′) complementary to Y2;
      C) a region (B′) upstream to Y2′; and
      D) an additional region (P′) between Y1′ and B′;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region (P), wherein P is downstream of B and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid upstream of a sequence complementary to at least a portion of region Y2 and primes the synthesis of a complementary nucleic acid strand while looping out region B of the extended target nucleic acid;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In one embodiment, the nucleic acid probe has a blocked 3′ end.

In another embodiment, the nucleic acid polymerase substantially lacks 5′ to 3′ exonuclease activity.

In still another embodiment, Y1′, Y2′, P′, and P comprise about 15-20 nucleotides in length.

In a preferred embodiment, the nuclease comprises a FEN nuclease. More preferably, it is selected from the group consisting of FEN nuclease enzyme derived from Archaeglobus fulgidus, Methanococcus jannaschii, and Pyrococcus furiosus. The nuclease may be derived from Taq, Tfl and Bca.

The invention also provides a method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
   i) the target nucleic acid comprises:
      A) a 3′ region (Y);
      B) a region (X) upstream of Y; and
      C) a series of intervening regions (A, Z1, Z2, B) between X and Y in the order of X-A-Z1-Z2-B-Y;
   ii) the nucleic acid probe comprises:
      A) a 5′ region (Z1′) complementary to Z1;
      B) a 3′ region (Z2′) complementary to Z2;
      C) a region (X′) complementary to X;
      D) a region (Y′) complementary to Y; and
      E) an additional region (P′) between X1′ and Y1′;
b) annealing the nucleic acid probe to the target nucleic acid and priming synthesis of an extended target nucleic acid while looping out one or more portions of regions A and B of the target sequence;
c) incubating the sample with a nucleic acid polymerase and extending the 3′ end of the target nucleic acid to generate an extended target nucleic acid having a primer binding region (P), wherein P is downstream of Y and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid upstream of a sequence complementary to at least a portion of region Z2 and primes the synthesis of a complementary nucleic acid strand while looping out one or more portions of regions Y and B of the extended target nucleic acid;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

In one embodiment, the nucleic acid probe has a blocked 3′ end.

In another embodiment, the nucleic acid polymerase substantially lacks 5′ to 3′ exonuclease activity.

In still another embodiment, Y1′, Y2′, P′, and P comprise about 15-20 nucleotides in length.

In a preferred embodiment, the nuclease comprises a FEN nuclease. More preferably, it is selected from the group consisting of FEN nuclease enzyme derived from Archaeglobus fulgidus, Methanococcus jannaschii, and Pyrococcus furiosus. The nuclease may be derived from Taq, Tfl and Bca.

The invention also provides a kit for generating a signal indicative of the presence of a target nucleic acid sequence of a target nucleic acid in a sample, comprising a nucleic acid polymerase, a FEN nuclease, a suitable buffer, a nucleic acid probe, wherein:
i) the target nucleic acid comprises:
   A) a 3′ region; and
   B) an upstream region; and
ii) the nucleic acid probe comprises:
   A) a 5′ region complementary to the upstream region on the target nucleic acid;
   B) a 3′ region complementary to the 3′ region on the target nucleic acid; and
   C) an additional region.

In one embodiment, the nucleic acid probe has a blocked 3′ end.

In another embodiment, the nucleic acid polymerase substantially lacks 5′ to 3′ exonuclease activity.

In still another embodiment, the nucleic acid polymerase is thermostable.

In yet another embodiment, the FEN nuclease is thermostable.

In another embodiment, the kit further comprises a primer, wherein the primer has a sequence that comprises at least a portion of the sequence of the additional region of the nucleic acid probe. Preferably, the primer is labeled.

Nucleases

Nucleases useful according to the invention include any enzyme that possesses 5′ endonucleolytic activity for example a DNA polymerase, e.g. DNA polymerase I from E. coli, and DNA polymerase from Thermus aquaticus (Taq), Thermus thermophilus (Tth), and Thermus flavus (Tfl). Nucleases useful according to the invention also include DNA polymerases with 5′-3′ exonuclease activity, including but not limited to eubacterial DNA polymerase I, including enzymes derived from Thermus species (Taq, Tfl, Tth, Tca (caldophilus), Thr (brockianus)), enzymes derived from Bacillus species (Bst, Bca, Magenta (full length polymerases, NOT N-truncated versions)), enzymes derived from Thermotoga species (Tma (maritima, Tne (neopolitana)) and E. coli DNA polymerase I. The term nuclease also embodies FEN nucleases. A nuclease useful according to the invention cannot cleave either a primer that is not hybridized to a target nucleic acid or a target nucleic acid that is not hybridized to a probe.

FEN-1 is an ˜40 kDa divalent metal ion-dependent exo- and endonuclease that specifically recognizes the backbone of a 5′ single-stranded flap strand and tracks down this arm to the cleavage site, which is located at the junction wherein the two strands of duplex DNA adjoin the single-stranded arm. Both the endo- and exonucleolytic activities show little sensitivity to the base at the most 5′ position at the flap or nick. Both FEN-1 endo- and exonucleolytic substrate binding and cutting are stimulated by an upstream oligonucleotide (flap adjacent strand or primer). This is also the case for E. coli pol I. The endonuclease activity of the enzyme is independent of the 5′ flap length, cleaving a 5′ flap as small as one nucleotide. The endonuclease and exonuclease activities are insensitive to the chemical nature of the substrate, cleaving both DNA and RNA.

Both the endo- and exonucleolytic activities are inhibited by concentrations of salts in the physiological range. The exonuclease activity is inhibited 50-fold at 50 mM NaCl as compared to 0 mM NaCl. The endonuclease activity is inhibited only sevenfold at 50 mM NaCl (Reviewed in Lieber 1997, supra).

Although a 5′-OH terminus is a good substrate for FEN-1 loading onto a 5′ flap substrate, it serves as a very poor substrate when part of a nick in an otherwise double stranded DNA structure. The electrostatic repulsion by the terminal phosphate is likely to favor breathing of the substrate into a pseudo-flap configuration, providing the active form of the substrate for FEN-1. Such an explanation would indicate a single active site and a single mechanism of loading of FEN-1 onto the 5′ ssDNA terminus of the flap or pseudo-flap configuration of the nick. Consistent with this model are observations that optimal activity at a nick requires very low Mg⁺² and monovalent salt concentrations, which destabilize base-pairing and would favor breathing of a nick to a flap. Higher Mg⁺² and monovalent salt concentrations would disfavor breathing and inhibit cutting of nicked or gapped structures that do require breathing to convert to a flap. Cleavage of stable flap structures is optimal at moderate Mg⁺² levels and does not decrease with increasing Mg⁺² concentration. This is because a flap substrate does not have to melt out base pairs to achieve its structure; hence, it is entirely insensitive to Mg⁺². Though the endonucleolytic activity decreases with monovalent salt, the decline is not nearly as sharp as that seen for the exonucleolytic activity. Furthermore, it has previously been shown that one-nucleotide flaps are efficient substrates. All of these observations are consistent with the fact that when FEN-1 has been interpreted to be functioning as an exonuclease, the size of the degradation products vary from one to several nucleotides in length. Breathing of nicks into flaps of varying length would be expected to vary with local sequence, depending on the G/C content. In summary, a nick breathing to form a transient flap means that the exonucleolytic activity of FEN-1 is the same as the endonucleolytic activity (Reviewed in Lieber, 1997, supra).

The endonuclease and exonuclease activities of FEN-1 cleave both DNA and RNA without requiring accessory proteins. At the replication fork, however, FEN-1 does interact with other proteins, including a DNA helicase and the proliferating cell nuclear antigen (PCNA), the processivity factor for DNA polymerases δ and ε. PCNA significantly stimulates FEN-1 endo- and exonucleolytic activity.

The FEN-1 enzymes are functionally related to several smaller bacteriophage 5′□3′ exonucleases such as T5 5′ exonuclease and T4 RNase H as well as to the larger eukaryotic nucleotide excision repair enzymes such as XPG, which also acts in the transcription-coupled repair of oxidative base damage. In eubacteria such as Escherichia coli and Thermus aquaticus, Okazaki processing is provided by the PolI 5′ to 3′ exonuclease domain. These bacterial and phage enzymes share two areas of limited sequence homology with FEN-1, which are termed the N (N-terminal) and I (intermediate) regions, with the residue similarities concentrated around seven conserved acidic residues. Based on crystal structures of T4 RNase H and T5 exonuclease as well as mutagenesis data, it has been proposed that these residues bind to two Mg⁺² ions that are required for affecting DNA hydrolysis; however, the role each metal plays in the catalytic cycle, which is subtly different for each enzyme, is not well understood (Reviewed in Hosfield et al., 1998b, supra).

The fen-1 genes encoding FEN-1 enzymes useful in the invention include murine fen-1, human fen-1, rat fen-1, Xenopus laevis fen-1, and fen-1 genes derived from four archaebacteria Archaeglobus fulgidus, Methanococcus jannaschii, Pyrococcus furiosus and Pyrococcus horikoshii. cDNA clones encoding FEN-1 enzymes have been isolated from human (GenBank Accession Nos.: NM 004111 and L37374), mouse (GenBank Accession No.: L26320), rat (GenBank Accession No.: AA819793), Xenopus laevis (GenBank Accession Nos.: U68141 and U64563), and P. furiosus (GenBank Accession No.: AF013497). The complete nucleotide sequence for P. horikoshii flap endonuclease has also been determined (GenBank Accession No.: AB005215). The FEN-1 family also includes the Saccharomyces cerevisiae RAD27 gene (GenBank Accession No.: Z28113 Y13137) and the Saccharomyces pombe RAD2 gene (GenBank Accession No.: X77041). The archaeal genome of Methanobacterium thermautotrophiculum has also been sequenced. Although the sequence similarity between FEN-1 and prokaryotic and viral 5′□3′ exonucleases is low, FEN-1s within the eukaryotic kingdom are highly conserved at the amino acid level, with the human and S. cerevisiae proteins being 60% identical and 78% similar. The three archaebacterial FEN-1 proteins are also, highly homologous to the eukaryotic FEN-1 enzymes (Reviewed in Matsui et al., 1999., J. Biol. Chem., 274:18297, Hosfield et al., 1998b, J. Biol. Chem., 273:27154 and Lieber, 1997, BioEssays, 19:233).

The sequence similarities in the two conserved nuclease domains (N-terminal or N and intermediate or I domains) between human and other FEN-1 family members are 92% (murine), 79% (S. cerevisiae), 77% (S. pombe), 72% (A. fulgidus), 76% (M. jannaschii), and 74% (P. furiosus).

FEN-1 specifically recognizes the backbone of a 5′ single-stranded flap strand and migrates down this flap arm to the cleavage site located at the junction between the two strands of duplex DNA and the single-stranded arm. If the strand upstream of the flap (sometimes called the flap adjacent strand or primer strand) is removed, the resulting structure is termed a pseudo-Y. This structure is cleaved by FEN-1, but at 20- to 100-fold lower efficiency. FEN-1 does not cleave 3′ single-stranded flaps. However, FEN-1 acting as an exonuclease will hydrolyze dsDNA substrates containing a gap or nick (Reviewed in Hosfield et al., 1998a, supra, Hosfield et al., 1999b, supra and Lieber 1997, supra). Exonucleolytically, FEN-1 acts at a nick and, with lower efficiency, at a gap or a recessed 5′ end on dsDNA. At gapped structures, the efficiency of FEN-1 binding and cutting decreases with increasing gap size up to approximately five nucleotides and then stabilizes at a level of cleavage that is equivalent to activity on a recessed 5′ end within dsDNA. Blunt dsDNA, recessed 3′ ends and ssDNA are not cleaved (Reviewed in Lieber 1997, supra).

FEN nucleases that are useful according to the invention have been isolated from a variety of organisms including human (GenBank Accession Nos.: NM 004111 and L37374), mouse (GenBank Accession No.: L26320), rat (GenBank Accession No.: AA819793), yeast (GenBank Accession No.: Z28113 Y13137 and GenBank Accession No.: X77041) and xenopus laevis (GenBank Accession Nos.: U68141 and U64563). Such enzymes can be cloned and overexpressed using conventional techniques well known in the art.

A FEN nuclease according to the invention is preferably thermostable. Thermostable FEN nucleases have been isolated and characterized from a variety of thermostable organisms including four archeaebacteria. The cDNA sequence (GenBank Accession No.: AF013497) and the amino acid sequence (Hosfield et al., 1998a, supra and Hosfield et al., 1998b) for P. furiosus flap endonuclease have been determined. The complete nucleotide sequence (GenBank Accession No.: AB005215) and the amino acid sequence (Matsui et al., supra) for P. horikoshii flap endonuclease have also been determined. The amino acid sequence for M. jannaschii (Hosfield et al., 1998b and Matsui et al., 1999 supra) and A. fulgidus (Hosfield et al., 1998b) flap endonuclease have also been determined.

Thermostable FEN-1 enzymes can be cloned and overexpressed using techniques well known in the art and described in Hosfield et al., 1998a, supra, Hosfield et al., 1998b, Kaiser et al., 1999, J. Biol. Chem., 274: 21387 and Matusi et al., supra and as described in U.S. Pat. No. 6,528,254 B1, filed Oct. 29, 1999; U.S. Pat. No. 6,548,250, filed Aug. 30, 2000 both of which are herein incorporated by reference in their entireties.

Nucleic Acid Polymerases

The invention provides for nucleic acid polymerases. Preferably, the nucleic acid polymerase according to the invention is thermostable.

Known DNA polymerases include, for example, E. coli DNA polymerase I, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

Nucleic acid polymerases substantially lacking 5′ to 3′ exonuclease activity useful according to the invention include but are not limited to Klenow and Klenow exo−, and T7 DNA polymerase (Sequenase).

Thermostable nucleic acid polymerases substantially lacking 5′ to 3′ exonuclease activity useful according to the invention include but are not limited to Pfu, exo− Pfu (a mutant form of Pfu that lacks 3′ to 5′ exonuclease activity), the Stoffel fragment of Taq, N-truncated Bst, N-truncated Bca, Genta, JdF3 exo−, Vent, Vent exo− (a mutant form of Vent that lacks 3′ to 5′ exonuclease activity), Deep Vent, Deep Vent exo− (a mutant form of Deep Vent that lacks 3′ to 5′ exonuclease activity), U1Tma, and ThermoSequenase.

Nucleic acid polymerases useful according to the invention include both native polymerases as well as polymerase mutants, which lack 5′ to 3′ exonuclease activity. Nucleic acid polymerases useful according to the invention can possess different degrees of thermostability. Preferably, a nucleic acid polymerase according to the invention exhibits strand displacement activity at the temperature at which it can extend a nucleic acid primer. In a preferred embodiment of the invention, a nucleic acid polymerase lacks both 5′ to 3′ and 3′ to 5′ exonuclease activity.

Additional nucleic acid polymerases substantially lacking 5′ to 3′ exonuclease activity with different degrees of thermostability useful according to the invention are listed below.

A. Bacteriophage DNA Polymerases (Useful for 37° C. Assays):

Bacteriophage DNA polymerases are devoid of 5′ to 3′ exonuclease activity, as this activity is encoded by a separate polypeptide. Examples of suitable DNA polymerases are T4, T7, and φ29 DNA polymerase. The enzymes available commercially are: T4 (available from many sources e.g., Epicentre) and T7 (available from many sources, e.g. Epicentre for unmodified and USB for 3′ to 5′ exo⁻ T7 “Sequenase” DNA polymerase).

B. Archaeal DNA Polymerases:

There are 2 different classes of DNA polymerases which have been identified in archaea: 1. Family B/pol α type (homologs of Pfu from Pyrococcus furiosus) and 2. pol II type (homologs of P. furiosus DP1/DP2 2-subunit polymerase). DNA polymerases from both classes have been shown to naturally lack an associated 5′ to 3′ exonuclease activity and to possess 3′ to 5′ exonuclease (proofreading) activity. Suitable DNA polymerases (pol α or pol II) can be derived from archaea with optimal growth temperatures that are similar to the desired assay temperatures. Examples of suitable archaea include, but are not limited to:

1. Thermolabile (useful for 37° C. assays)—e.g., Methanococcus voltae

2. Thermostable (useful for non-PCR assays)—e.g., Sulfolobus solfataricus, Suljolobus acidocaldarium, Methanococcus jannaschi, Thermoplasma acidophilum. It is estimated that suitable archaea exhibit maximal growth temperatures of ≦80-85° C. or optimal growth temperatures of ≦70-80° C.

3. Thermostable (useful for PCR assays)—e.g., Pyrococcus species (furiosus, species GB-D, species strain KOD1, woesii, abysii, horikoshii), Thermococcus species (litoralis, species 90North-7, species JDF-3, gorgonarius), Pyrodictium occultum, and Archaeoglobus fulgidus. It is estimated that suitable archaea would exhibit maximal growth temperatures of ≧80-85° C. or optimal growth temperatures of ≧70-80° C. Appropriate PCR enzymes from the archaeal pol a DNA polymerase group are commercially available, including KOD (Toyobo), Pfx (Life Technologies, Inc.), Vent (New England BioLabs), Deep Vent (New England BioLabs), and Pwo (Boehringer-Mannheim).

Additional archaea related to those listed above are described in the following references: Archaea: A Laboratory Manual (Robb, F. T. and Place, A. R., eds.), Cold Spring Harbor Laboratory Press, Cold Spring Harbor, N.Y., 1995 and Thermophilic Bacteria (Kristjansson, J. K., ed.) CRC Press, Inc., Boca Raton, Fla., 1992.

C. Eubacterial DNA Polymerases:

There are 3 classes of eubacterial DNA polymerases, pol I, II, and III. Enzymes in the Pol I DNA polymerase family possess 5′ to 3′ exonuclease activity, and certain members also exhibit 3′ to 5′ exonuclease activity. Pol II DNA polymerases naturally lack 5′ to 3′ exonuclease activity, but do exhibit 3′ to 5′ exonuclease activity. Pol III DNA polymerases represent the major replicative DNA polymerase of the cell and are composed of multiple subunits. The pol III catalytic subunit lacks 5′ to 3′ exonuclease activity, but in some cases 3′ to 5′ exonuclease activity is located in the same polypeptide.

There are a variety of commercially available Pol I DNA polymerases, some of which have been modified to reduce or abolish 5′ to 3′ exonuclease activity. Methods used to eliminate 5′ to 3′ exonuclease activity of pol I DNA polymerases include:
mutagenesis (as described in Xu et al., 1997, J. Mol. Biol., 268:284 and Kim et al., 1997, Mol. Cells, 7:468).
N-truncation by proteolytic digestion (as described in Klenow et al., 1971, Eur. J. Biochem., 22: 371), or
N-truncation by cloning and expressing as C-terminal fragments (as described in Lawyer et al., 1993, PCR Methods Appl., 2:275).

As for archaeal sources, the assay-temperature requirements determine which eubacteria should be used as a source of a DNA polymerase useful according to the invention (e.g., mesophiles, thermophiles, hyperthermophiles).

1. Mesophilic/Thermolabile (Useful for 37° C. Assays)
i. DNA polymerases naturally substantially lacking 5′ to 3′ exonuclease activity: pol II or the pol III catalytic subunit from mesophilic eubacteria, such as Escherichia coli, Streptococcus pneumoniae, Haemophilus influenza, Mycobacterium species (tuberculosis, leprae)
ii. DNA polymerase mutants substantially lacking 5′ to 3′ exonuclease activity: Pol I DNA polymerases for N-truncation or mutagenesis can be isolated from the mesophilic eubacteria listed above (Ci). A commercially-available eubacterial DNA polymerase pol I fragment is the Klenow fragment (N-truncated E. coli pol I; Stratagene).

2. Thermostable (Useful for Non PCR Assays)
i. DNA polymerases naturally substantially lacking 5′ to 3′ exonuclease activity: Pol II or the pol III catalytic subunit from thermophilic eubacteria, such as Bacillus species (e.g., stearothermophilus, caldotenax, caldovelox)
ii. DNA polymerase mutants substantially lacking 5′ to 3′ exonuclease activity: Suitable pol I DNA polymerases for N-truncation or mutagenesis can be isolated from thermophilic eubacteria such as the Bacillus species listed above. Thermostable N-truncated fragments of B. stearothermophilus DNA polymerase pol I are commercially available and sold under the trade names Bst DNA polymerase I large fragment (Bio-Rad and Isotherm DNA polymerase (Epicentre)). A C-terminal fragment of Bacillus caldotenax pol I is available from Panvera (sold under the tradename Ladderman).

3. Thermostable (Useful for PCR Assays)
i. DNA polymerases naturally substantially lacking 5′ to 3′ exonuclease activity: Pol II or pol III catalytic subunit from Thermus species (aquaticus, thermophilus, flavus, ruber, caldophilus, filiformis, brokianus) or from Thermotoga maritima. The catalytic pol III subunits from Thermus thermophilus and Thermus aquaticus are described in Yi-Ping et al., 1999, J. Mol. Evol., 48:756 and McHenry et al., 1997, J. Mol. Biol., 272:178.
ii. DNA polymerase mutants substantially lacking 5′ to 3′ exonuclease activity: Suitable pol I DNA polymerases for N-truncation or mutagenesis can be isolated from a variety of thermophilic eubacteria, including Thermus species and Thermotoga maritima (see above). Thermostable fragments of Thermus aquaticus DNA polymerase pol I (Taq) are commercially available and sold under the trade names KlenTaq1 (Ab Peptides), Stoffel fragment (Perkin-Elmer), and ThermoSequenase (Amersham). In addition to C-terminal fragments, 5′ to 3′ exonuclease⁻ Taq mutants are also commercially available, such as TaqFS (Hoffman-LaRoche). In addition to 5′-3′ exonuclease⁻ versions of Taq, an N-truncated version of Thermotoga maritima DNA polymerase I is also commercially available (tradename UlTma, Perkin-Elmer).

Additional eubacteria related to those listed above are described in Thermophilic Bacteria (Kristjansson, J. K., ed.) CRC Press, Inc., Boca Raton, Fla., 1992.

D. Eukaryotic 5′ to 3′ Exonuclease⁻ DNA Polymerases (Useful for 37° C. Assays)

There are several DNA polymerases that have been identified in eukaryotes, including DNA pol α (replication/repair), δ (replication), ε (replication), β (repair) and γ (mitochondrial replication). Eukaryotic DNA polymerases are devoid of 5′ to 3′ exonuclease activity, as this activity is encoded by a separate polypeptide (e.g., mammalian FEN-1 or yeast RAD2). Suitable thermolabile DNA polymerases may be isolated from a variety of eukaryotes (including but not limited to yeast, mammalian cells, insect cells, Drosophila) and eukaryotic viruses (e.g., EBV, adenovirus).

It is possible that DNA polymerase mutants lacking 3′-5′ exonuclease (proofreading) activity, in addition to lacking 5′ to 3′ exonuclease activity, could exhibit improved performance in FEN-based detection strategies. For example, reducing or abolishing inherent 3′ to 5′ exonuclease activity may lower background signals by diminishing non-specific exonucleolytic degradation of labeled probes. Three 3′ to 5′ exonuclease motifs have been identified, and mutations in these regions have been shown to abolish 3′ to 5′ exonuclease activity in Klenow, φ29, T4, T7, and Vent DNA polymerases, yeast Pol α, Pol β, and Pol γ, and Bacillus subtilis Pol III (reviewed in Derbeyshire et al., 1995, Methods. Enzymol. 262:363). Methods for preparing additional DNA polymerase mutants, with reduced or abolished 3′ to 5′ exonuclease activity, are well known in the art.

Commercially-available enzymes that lack both 5′ to 3′ and 3′ to 5′ exonuclease activities include Sequenase (exo⁻ T7; USB), Pfu exo⁻ (Stratagene), exo⁻ Vent (New England BioLabs), exo⁻ DeepVent (New England BioLabs), exo⁻ Klenow fragment (Stratagene), Bst (Bio-Rad), Isotherm (Epicentre), Ladderman (Panvera), KlenTaq1 (Ab Peptides), Stoffel fragment (Perkin-Elmer), ThermoSequenase (USB), and TaqFS (Hoffman-LaRoche).

If polymerases other than Pfu are used, buffers and extension temperatures are selected to allow for optimal activity by the particular polymerase useful according to the invention. Buffers and extension temperatures useful for polymerases according to the invention are know in the art and can also be determined from the Vendor's specifications.

Nucleic Acids

A. Nucleic Acid Sequences Useful in the Invention

The invention provides for methods of detecting or measuring a target nucleic acid sequence; and also utilizes oligonucleotides, primers and probes for forming a cleavage structure according to the invention and primers for amplifying a template nucleic acid sequence. As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including a basic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

The complement of a nucleic acid sequence as used herein refers to an oligonucleotide which, when aligned with the nucleic acid sequence such that the 5′ end of one sequence is paired with the 3′ end of the other, is in “antiparallel association.”

The oligonucleotide is not necessarily physically derived from any existing or natural sequence but may be generated in any manner, including chemical synthesis, DNA replication, reverse transcription or a combination thereof. The terms “oligonucleotide” or “nucleic acid” intend a polynucleotide of genomic DNA or RNA, cDNA, semisynthetic, or synthetic origin which, by virtue of its synthetic origin or manipulation: (1) is not associated with all or a portion of the polynucleotide with which it is associated in nature; and/or (2) is linked to a polynucleotide other than that to which it is linked in nature.

Because mononucleotides are reacted to make oligonucleotides in a manner such that the 5′ phosphate of one mononucleotide pentose ring is attached to the 3′ oxygen of its neighbor in one direction via a phosphodiester linkage, an end of oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

When two different, non-overlapping oligonucleotides anneal to different regions of the same linear complementary nucleic acid sequence, and the 3′ end of one oligonucleotide points toward the 5′ end of the other, the former may be called the “upstream” oligonucleotide and the latter the “downstream” oligonucleotide.

Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the base pairs have disassociated.

B. Production of a Nucleic Acid

The invention provides nucleic acids to be detected and or measured, for amplification of a target nucleic acid sequence and for formation of a cleavage structure.

The present invention utilizes nucleic acids comprising RNA, cDNA, genomic DNA, synthetic forms, and mixed polymers. The invention includes both sense and antisense strands of a nucleic acid. According to the invention, the nucleic acid may be chemically or biochemically modified or may contain non-natural or derivatized nucleotide bases. Such modifications include, for example, labels, methylation, substitution of one or more of the naturally occurring nucleotides with an analog, internucleotide modifications such as uncharged linkages (e.g. methyl phosphonates, phosphorodithioates, etc.), pendent moieties (e.g., polypeptides), intercalators, (e.g. acridine, psoralen, etc.) chelators, alkylators, and modified linkages (e.g. alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

1. Nucleic Acids Comprising DNA

a. Cloning

Nucleic acids comprising DNA can be isolated from cDNA or genomic libraries by cloning methods well known to those skilled in the art (Ausubel et al., supra). Briefly, isolation of a DNA clone comprising a particular nucleic acid sequence involves screening a recombinant DNA or cDNA library and identifying the clone containing the desired sequence. Cloning will involve the following steps. The clones of a particular library are spread onto plates, transferred to an appropriate substrate for screening, denatured, and probed for the presence of a particular nucleic acid. A description of hybridization conditions, and methods for producing labeled probes is included below.

The desired clone is preferably identified by hybridization to a nucleic acid probe or by expression of a protein that can be detected by an antibody. Alternatively, the desired clone is identified by polymerase chain amplification of a sequence defined by a particular set of primers according to the methods described below.

The selection of an appropriate library involves identifying tissues or cell lines that are an abundant source of the desired sequence. Furthermore, if a nucleic acid of interest contains regulatory sequence or intronic sequence a genomic library is screened (Ausubel et al., supra).

b. Genomic DNA

Nucleic acid sequences of the invention are amplified from genomic DNA. Genomic DNA is isolated from tissues or cells according to the following method.

To facilitate detection of a variant form of a gene from a particular tissue, the tissue is isolated free from surrounding normal tissues. To isolate genomic DNA from mammalian tissue, the tissue is minced and frozen in liquid nitrogen. Frozen tissue is ground into a fine powder with a prechilled mortar and pestle, and suspended in digestion buffer (100 mM NaCl, 10 mM Tris-HCl, pH 8.0, 25 mM EDTA, pH 8.0, 0.5% (w/v) SDS, 0.1 mg/ml proteinase K) at 1.2 ml digestion buffer per 100 mg of tissue. To isolate genomic DNA from mammalian tissue culture cells, cells are pelleted by centrifugation for 5 min at 500×g, resuspended in 1-10 ml ice-cold PBS, repelleted for 5 min at 500×g and resuspended in 1 volume of digestion buffer.

Samples in digestion buffer are incubated (with shaking) for 12-18 hours at 50° C., and then extracted with an equal volume of phenol/chloroform/isoamyl alcohol. If the phases are not resolved following a centrifugation step (10 min at 1700×g), another volume of digestion buffer (without proteinase K) is added and the centrifugation step is repeated. If a thick white material is evident at the interface of the two phases, the organic extraction step is repeated. Following extraction the upper, aqueous layer is transferred to a new tube to which will be added ½ volume of 7.5M ammonium acetate and 2 volumes of 100% ethanol. The nucleic acid is pelleted by centrifugation for 2 min at 1700×g, washed with 70% ethanol, air dried and resuspended in TE buffer (10 mM Tris-HCl, pH 8.0, 1 mM EDTA, pH 8.0) at 1 mg/ml. Residual RNA is removed by incubating the sample for 1 hour at 37° C. in the presence of 0.1% SDS and 1 μg/ml DNase-free RNase, and repeating the extraction and ethanol precipitation steps. The yield of genomic DNA, according to this method is expected to be approximately 2 mg DNA/1 g cells or tissue (Ausubel et al., supra). Genomic DNA isolated according to this method can be used for PCR analysis, according to the invention.

c. Restriction Digest (of cDNA or Genomic DNA)

Following the identification of a desired cDNA or genomic clone containing a particular target nucleic acid sequence, nucleic acids of the invention may be isolated from these clones by digestion with restriction enzymes.

The technique of restriction enzyme digestion is well known to those skilled in the art (Ausubel et al., supra). Reagents useful for restriction enzyme digestion are readily available from commercial vendors including Stratagene, as well as other sources.

d. PCR

Nucleic acids of the invention may be amplified from genomic DNA or other natural sources by the polymerase chain reaction (PCR). PCR methods are well-known to those skilled in the art.

PCR provides a method for rapidly amplifying a particular DNA sequence by using multiple cycles of DNA replication catalyzed by a thermostable, DNA-dependent DNA polymerase to amplify the target sequence of interest. PCR requires the presence of a target nucleic acid sequence to be amplified, two single stranded oligonucleotide primers flanking the sequence to be amplified, a DNA polymerase, deoxyribonucleoside triphosphates, a buffer and salts.

PCR, is performed as described in Mullis and Faloona, 1987, Methods Enzymol., 155: 335, herein incorporated by reference.

The polymerase chain reaction (PCR) technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment whose termini are defined by the 5′ ends of the primers. PCR is reported to be capable of producing a selective enrichment of a specific DNA sequence by a factor of 10⁹. The PCR method is also described in Saiki et al., 1985, Science 230:1350.

PCR is performed using template DNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of a suitable buffer, 0.4 μl of 1.25 μM dNTP, 2.5 units of Taq DNA polymerase (Stratagene) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

Detection methods generally employed in standard PCR techniques use a labeled probe with the amplified DNA in a hybridization assay. Preferably, the probe is labeled, e.g., with ³²P, biotin, horseradish peroxidase (HRP), etc., to allow for detection of hybridization.

Other means of detection include the use of fragment length polymorphism (PCR FLP), hybridization to allele-specific oligonucleotide (ASO) probes (Saiki et al., 1986, Nature 324:163), or direct sequencing via the dideoxy method (using amplified DNA rather than cloned DNA). The standard PCR technique operates (essentially) by replicating a DNA sequence positioned between two primers, providing as the major product of the reaction a DNA sequence of discrete length terminating with the primer at the 5′ end of each strand. Thus, insertions and deletions between the primers result in product sequences of different lengths, which can be detected by sizing the product in PCR-FLP. In an example of ASO hybridization, the amplified DNA is fixed to a nylon filter (by, for example, UV irradiation) in a series of “dot blots”, then allowed to hybridize with an oligonucleotide probe labeled with HRP under stringent conditions. After washing, terramethylbenzidine (TMB) and hydrogen peroxide are added: HRP oxidizes the hydrogen peroxide, which in turn oxidizes the TMB to a blue precipitate, indicating a hybridized probe.

A PCR assay for detecting or measuring a nucleic assay according to the invention is described in the section entitled “Methods of Use”.

2. Nucleic Acids Comprising RNA

The present invention also provides a nucleic acid comprising RNA.

Nucleic acids comprising RNA can be purified according to methods well known in the art (Ausubel et al., supra). Total RNA can be isolated from cells and tissues according to methods well known in the art (Ausubel et al., supra) and described below.

RNA is purified from mammalian tissue according to the following method. Following removal of the tissue of interest, pieces of tissue of ≦2 g are cut and quick frozen in liquid nitrogen, to prevent degradation of RNA. Upon the addition of a suitable volume of guanidinium solution (for example 20 ml guanidinium solution per 2 g of tissue), tissue samples are ground in a tissuemizer with two or three 10-second bursts. To prepare tissue guanidinium solution (1 L) 590.8 g guanidinium isothiocyanate is dissolved in approximately 400 ml DEPC-treated H₂O. 25 ml of 2 M Tris-HCl, pH 7.5 (0.05 M final) and 20 ml Na₂EDTA (0.01 M final) is added, the solution is stirred overnight, the volume is adjusted to 950 ml, and 50 ml 2-ME is added.

Homogenized tissue samples are subjected to centrifugation for 10 min at 12,000×g at 12° C. The resulting supernatant is incubated for 2 min at 65° C. in the presence of 0.1 volume of 20% Sarkosyl, layered over 9 ml of a 5.7M CsCl solution (0.1 g CsCl/ml), and separated by centrifugation overnight at 113,000×g at 22° C. After careful removal of the supernatant, the tube is inverted and drained. The bottom of the tube (containing the RNA pellet) is placed in a 50 ml plastic tube and incubated overnight (or longer) at 4° C. in the presence of 3 ml tissue resuspension buffer (5 mM EDTA, 0.5% (v/v) Sarkosyl, 5% (v/v) 2-ME) to allow complete resuspension of the RNA pellet. The resulting RNA solution is extracted sequentially with 25:24:1 phenol/chloroform/isoamyl alcohol, followed by 24:1 chloroform/isoamyl alcohol, precipitated by the addition of 3 M sodium acetate, pH 5.2, and 2.5 volumes of 100% ethanol, and resuspended in DEPC water (Chirgwin et al., 1979, Biochemistry, 18: 5294).

Alternatively, RNA is isolated from mammalian tissue according to the following single step protocol. The tissue of interest is prepared by homogenization in a glass Teflon homogenizer in 1 ml denaturing solution (4M guanidinium thiosulfate, 25 mM sodium citrate, pH 7.0, 0.1M 2-ME, 0.5% (w/v) N-laurylsarkosine) per 100 mg tissue. Following transfer of the homogenate to a 5-ml polypropylene tube, 0.1 ml of 2 M sodium acetate, pH 4, 1 ml water-saturated phenol, and 0.2 ml of 49:1 chloroform/isoamyl alcohol are added sequentially. The sample is mixed after the addition of each component, and incubated for 15 min at 0-4° C. after all components have been added. The sample is separated by centrifugation for 20 min at 10,000×g, 4° C., precipitated by the addition of 1 ml of 100% isopropanol, incubated for 30 minutes at −20° C. and pelleted by centrifugation for 10 minutes at 10,000×g, 4° C. The resulting RNA pellet is dissolved in 0.3 ml denaturing solution, transferred to a microfuge tube, precipitated by the addition of 0.3 ml of 100% isopropanol for 30 minutes at −20° C., and centrifuged for 10 minutes at 10,000×g at 4° C. The RNA pellet is washed in 70% ethanol, dried, and resuspended in 100-200 μl DEPC-treated water or DEPC-treated 0.5% SDS (Chomczynski and Sacchi, 1987, Anal. Biochem., 162:156).

Nucleic acids comprising RNA can be produced according to the method of in vitro transcription.

The technique of in vitro transcription is well known to those of skill in the art. Briefly, the gene of interest is inserted into a vector containing an SP6, T3 or T7 promoter. The vector is linearized with an appropriate restriction enzyme that digests the vector at a single site located downstream of the coding sequence. Following a phenol/chloroform extraction, the DNA is ethanol precipitated, washed in 70% ethanol, dried and resuspended in sterile water. The in vitro transcription reaction is performed by incubating the linearized DNA with transcription buffer (200 mM Tris-HCl, pH 8.0, 40 mM MgCl₂, 10 mM spermidine, 250 NaCl [T7 or T3] or 200 mM Tris-HCl, pH 7.5, 30 mM MgCl₂, 10 mM spermidine [SP6]), dithiothreitol, RNase inhibitors, each of the four ribonucleoside triphosphates, and either SP6, T7 or T3 RNA polymerase for 30 min at 37° C. To prepare a radiolabeled polynucleotide comprising RNA, unlabeled UTP will be omitted and ³⁵S-UTP will be included in the reaction mixture. The DNA template is then removed by incubation with DNaseI. Following ethanol precipitation, an aliquot of the radiolabeled RNA is counted in a scintillation counter to determine the cpm/μl (Ausubel et al., supra).

Alternatively, nucleic acids comprising RNA are prepared by chemical synthesis techniques such as solid phase phosphoramidite (described above).

3. Nucleic Acids Comprising Oligonucleotides

A nucleic acid comprising oligonucleotides can be made by using oligonucleotide synthesizing machines which are commercially available (described above).

C. Target Nucleic Acid

The present invention contemplates the detection and/or quantitation of a nucleic acid. The methods described herein can detect a very broad range of nucleic acids. In many circumstances, the nucleic acid can be detected directly; however, in other instances, the nucleic acid can be processed prior to the detection step. The target nucleic acid can be generated by any number of means. For example, it can be generated from a cleavage reaction by a restriction enzyme or other endo- or exonucleases. Alternatively, it can form as a result of a specific or non-specific cleavage of a longer nucleic acid strand, and can be generated enzymatically or chemically. In a preferred embodiment, the target nucleic acid is generated by PCR. The target nucleic acid of the present invention also contemplates fragments generated by aged tissue, apoptotic cells, or the consequence of any other natural, biological or chemical reaction that may generate nucleic acid fragments.

According to the present invention, a target nucleic acid is a nucleic acid whose presence needs to be determined, for example, in a sample. The target nucleic acid can be of any length greater than 10 bases in length, for example 20, 25, 30, 40, 50, 60, 100 bases in length or more. The target nucleic acid is typically single stranded. However, the presence of a double stranded nucleic acid can also readily be detected by first denaturing the sample, then using one of the two strands as a target nucleic acid for determination. Alternatively, if the double stranded nucleic acid whose presence is to be determined contains a single stranded region, this region can be used as sites of hybridization with the target nucleic acid binding sites of the probe.

The target nucleic acid can be composed of natural, non-natural nucleic acids or combinations thereof. The only requirement of the target nucleic acid is that the 3′ end of the target nucleic acid must support polymerase dependent extension reactions (i.e., must not be blocked), and that the 5′ end must be ligatable. Thus, while it is not necessary that the entire sequence of the target nucleic acid be known, the sequence of the 5′ end and 3′ end of the target nucleic acid must be known. The 5′ end and 3′ end of the target nucleic acid contain probe interacting sites which are complementary to the target nucleic acid binding sites of the probe.

In certain embodiments, the nucleic acid whose presence is to be determined is not ideal for detection using the methods described herein, for example, because one the ends is blocked or damaged or the nucleic acid is circular. It will be appreciated by one of skill in the art that such nucleic acids can be converted to a target nucleic acid by processing the nucleic acid either enzymatically or chemically. For example, a circular nucleic acid can be linearized using specific or non-specific nucleases at specified conditions, and blocked ends preventing extension can likewise be removed by limited treatment with exonucleases. In addition, it will also be apparent to one of skill in the art that detection of long nucleic acids (i.e., greater than 10 kb) can also be performed, for example, by generating a smaller fragment by restriction endonucleases.

Probe

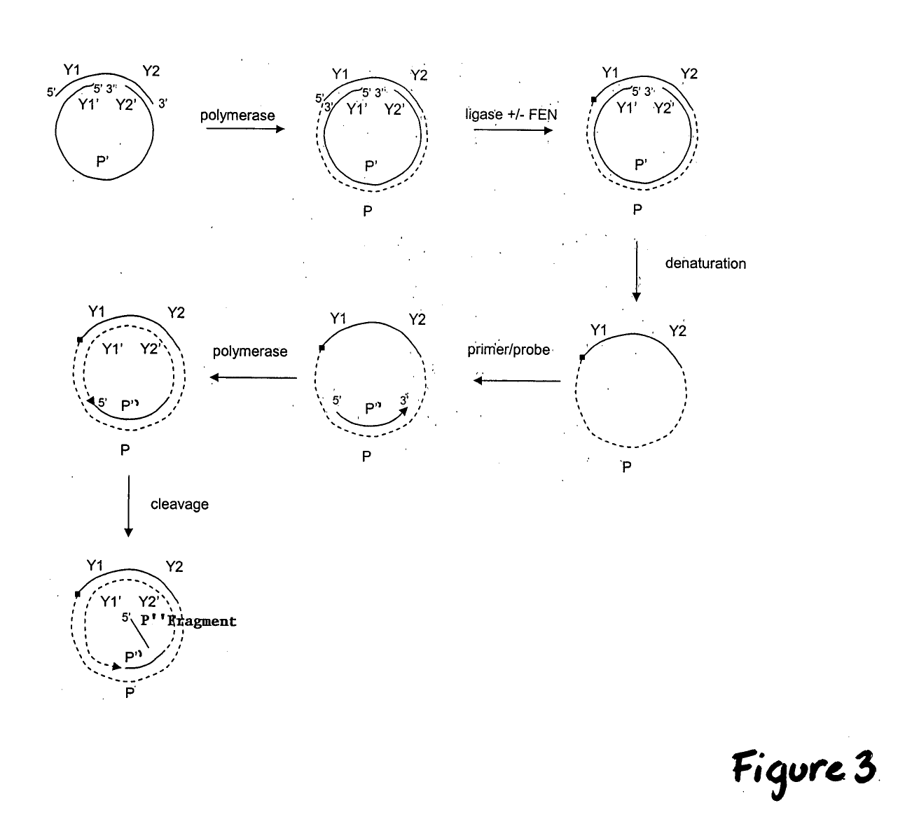

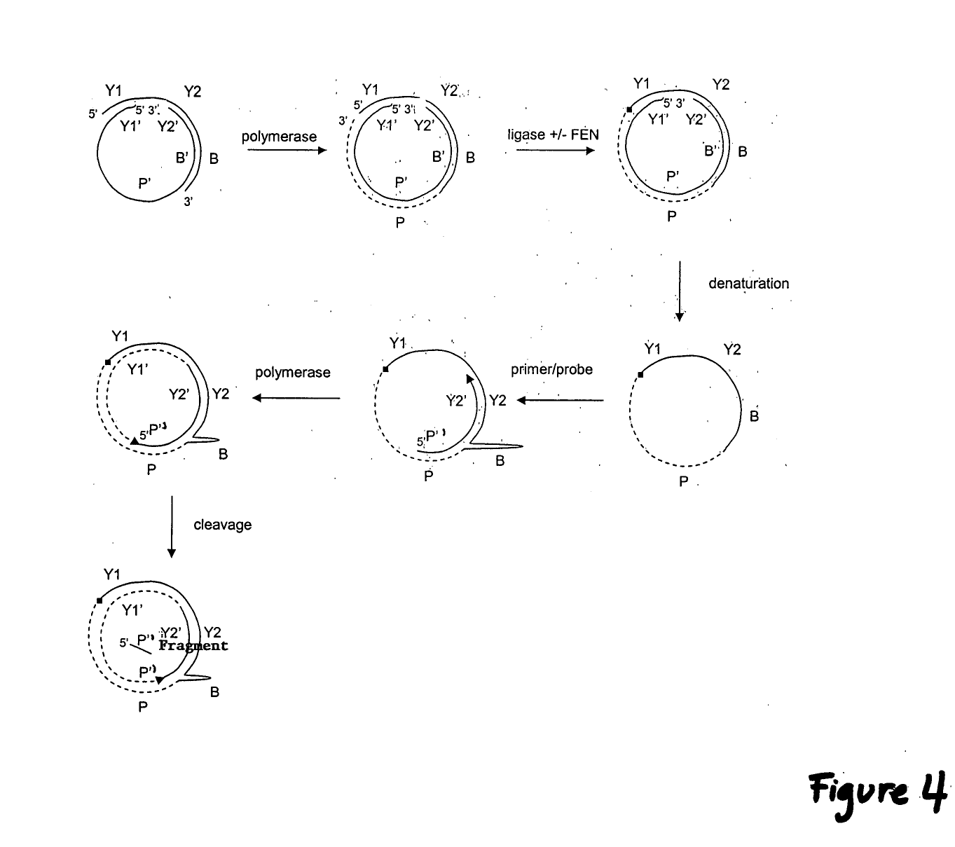

According to the present invention, the probe has a 5′ end and a 3′ end. The probe further comprises a first target nucleic acid binding site and a second target nucleic acid binding site (For example, Y1′ and Y2′ of FIG. 3). The probe is blocked from its 3′ end. Furthermore, in one embodiment the probe is not ligatable from its 5′ end. The probe comprises two target nucleic acid binding sites, each capable of binding to a different region of the target nucleic acid. The probe is capable of forming a circular hybridization complex with the target nucleic acid.

The probe of the present invention can be of any length, so long as it is capable of forming a hybridization complex with the target nucleic acid (e.g., it must contain two target nucleic acid binding sites), and also allowing for effective extension reaction from the end of the target nucleic acid. In one embodiment, the probe is between 30 and 1 kb bases in length. In another embodiment, the probe is between 100 and 200 bases in length.

The probe can comprise natural or non-natural nucleic acids, or combinations thereof. The probe can comprise a nucleic acid analog or chimera comprising nucleic acid and nucleic acid analog monomer units, such as 2-aminoethylglycine, a peptide nucleic acid (PNA), or a locked nucleic acid (LNA). For example, part or all of the oligonucleotide may be LNA or a LNA/nucleic acid (DNA or RNA) chimera. In one embodiment, the oligonucleotide comprises at least one locked nucleic acid. Locked nucleic acids represent a class of conformationally restricted nucleotide analogues described, for example, in WO 99/14226, which is incorporated by reference. LNAs hybridize more strongly to both DNA and RNA than naturally occurring nucleotides. Oligonucleotides containing the locked nucleotide are described in Koshkin, A. A., et al., Tetrahedron (1998), 54: 3607-3630) and Obika, S. et al., Tetrahedron Lett. (1998), 39: 5401-5404), both of which are incorporated by reference. Introduction of a locked nucleotide into an oligonucleotide improves the affinity for complementary sequences and increases the melting temperature by several degrees (Braasch, D. A. and D. R. Corey, Chem. Biol. (2001), 8:1-7). The invention can be carried out with any of the LNAs known in the art, for example, those disclosed in WO 99/14226 and in Latorra D, et al., 2003. Hum. Mutat. 22: 79-85, both of which are incorporated herein by reference. More specific binding can be obtained and more stringent washing conditions can be employed using LNA analogs, with the advantage that the amount of background noise is reduced significantly.

The probe comprises a first target nucleic acid binding site and a second target nucleic acid binding site. Target nucleic acid binding sites are sequences within the probe which are complementary to a sequence within the target nucleic acid. The first and second target nucleic acid binding sequences are complementary to two different regions within the target nucleic acid. The target nucleic acid binding sites are generally between 10 and 40 bases in length. In one embodiment, the target nucleic acid binding site is between 15 and 25 bases in length. The target nucleic acid binding sequences can be positioned at the 3′ or 5′ termini (i.e., 3′ or 5′ ends). Alternatively, the target nucleic acid binding sites can be positioned anywhere within the probe, so long as the probe and the target nucleic acid can form a circular hybridization complex. In one embodiment, the first target nucleic acid binding site is at either of the 5′ end or 3′ end of the probe. In another embodiment, the second target nucleic acid binding site is located at the other of the 5′ end or 3′ end of the probe. In yet another embodiment, the first target nucleic acid binding site which hybridizes to a site at the 3′ end of the target nucleic acid is located at or near the 5′ end of the probe, and the second target nucleic acid binding site which hybridizes to a site at the 5′ end of the target nucleic acid is located at or near the 3′ end of the probe.

The probe is capable of forming a hybridization complex with the target nucleic acid. The hybridization complex is formed through hybridization of the target nucleic acid with the probe at the two target nucleic acid binding sites, each of which is complementary to different portions within the target nucleic acid. An example of a hybridization complex is shown in FIGS. 1 and 3-5.

In some embodiments, the probe according to the present invention does not support an extension reaction from its 3′ end. In one embodiment, the 3′ end nucleotide of the probe is blocked. For example, the 3′ end nucleotide can be a dideoxy nucleic acid or any other nucleic acid (natural or otherwise) which does not allow extension using a polymerase enzyme. In another embodiment, a chemical moiety is attached to the 3′ end of the probe, for example, on the 3′ OH moiety, to prevent extension from that end. In still another embodiment, the 3′ end of the probe does not hybridize with a primer, such that the 3′ end of the probe cannot serve as a primer for an extension reaction by a polymerase.

In one embodiment, the probe also comprises a first primer binding region. In another embodiment, the probe further comprises a second primer binding region. In general, the primer binding region does not overlap with the target nucleic acid binding site of the probe. Furthermore, in embodiments encompassing a first primer binding region and a second primer binding region, the two primer binding regions generally do not overlap with each other. In another embodiment, the second primer binding region is located between the first and second target nucleic acid binding sequence of the probe. The primer binding region can be located at least 15 bases from one of the target nucleic acid binding sites, for example at least 20, 25, 30, 35, 40, 45 bases or more from one of the target nucleic acid binding sites. In still another embodiment, the probe comprises two primer binding regions, each of which is positioned between the first and second target nucleic acid binding region, and separated by at least 20 bases from one of the target nucleic acid binding sites. In another embodiment, they are separated by at least 30 bases from one of the target nucleic acid binding sites

A wide range of fluorophores may be used in probes and primers according to this invention. Available fluorophores include coumarin, fluorescein, tetrachlorofluorescein, hexachlorofluorescein, Lucifer yellow, rhodamine, BODIPY, tetramethylrhodamine, Cy3, Cy5, Cy7, eosine, Texas red and ROX. Combination fluorophores such as fluorescein-rhodamine dimers, described, for example, by Lee et al. (1997), Nucleic Acids Research 25:2816, are also suitable. Fluorophores may be chosen to absorb and emit in the visible spectrum or outside the visible spectrum, such as in the ultraviolet or infrared ranges.

A quencher is a moiety that, when placed very close to an excited fluorophore, causes there to be little or no fluorescence. Suitable quenchers described in the art include particularly DABCYL and variants thereof, such as DABSYL, DABMI and Methyl Red. Fluorophores can also be used as quenchers, because they tend to quench fluorescence when touching certain other fluorophores. Preferred quenchers are either chromophores such as DABCYL or malachite green, or fluorophores that do not fluoresce in the detection range when the probe is in the open conformation.

Methods of labeling a probe according to the invention and suitable labels are well known in the art and described herein.

Methods of Use

A. Detection of Target Nucleic Acids

As described herein, the method of detecting the target nucleic acid comprises the following steps:
a) extending the target nucleic acid to add an additional region;
b) circularizing the extended target nucleic acid;
c) forming a cleavage structure by incubating the sample with a primer which hybridizes to the circularized and extended target nucleic acid and extending the 3′ end of the primer with a nucleic acid polymerase and displacing the 5′ end of the primer;
d) cleaving the cleavage structure with a nuclease to release a nucleic acid fragment from the cleavage structure; and
e) detecting and/or measuring the release of the nucleic acid fragment as an indication of the presence of the target nucleic acid.

As previously described, the probe contains two target nucleic acid binding sites which are complementary to and hybridize to the target nucleic acid. The ideal Tₘ of the hybridization complex between the target nucleic acid and the probe is between 40° C. and 75° C., typically between 45° C. and 70° C., for example, between 50° C. and 65° C.

The equation for calculating the Tₘ of polynucleotides is well known in the art. For example, the Tₘ may be calculated by the following equation: Tₘ=69.3+0.41×(G+C)%−650/L, wherein L is the length of the oligonucleotide in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically. The stability and melting temperature of sequences can also be determined, for example, using programs such as mfold (Zuker (1989) Science, 244, 48-52) or Oligo 5.0 (Rychlik & Rhoads (1989) Nucleic Acids Res. 17, 8543-51). Methods for calculating the Tₘ of natural and non-natural nucleic acids are also known in the art. For example, the melting temperature LNA-DNA hybrids can be calculated using methods known in the art, for example as described in McTigue et al. (2004) Biochemistry, 43, 5388-5405, Tolstrup et al., (2003) Nucl. Acid Res. 31, 3758-62, incorporated by reference.

B. Extending and Circularizing

Once a circular hybridization complex is formed, the target nucleic acid is used as a primer in a template-dependent polymerization reaction. The extension step is carried out by providing a polymerase under conditions supporting polymerization (nucleotides, buffer, magnesium, and temperature appropriate for the given polymerase) known to one skilled in the art. The present invention contemplates the use of any polymerase known in the art. In one embodiment, the polymerase is a DNA polymerase. In another embodiment, the polymerase is an exonuclease deficient DNA polymerase. In still another embodiment, the polymerase is a thermostable DNA polymerase. In some embodiments, the extension reaction is performed under conditions in which strand displacement does not occur.

Once extended (See, for example, FIGS. 3-5), a covalent circular target nucleic acid is then formed by ligating the extension product to the 5′ end of the target nucleic acid. The 3′ extension of the target nucleic acid synthesizes a primer binding region (P region) which is complementary to the P′ region of the probe.

According to methods described herein, a probe is provided in concentrations exceeding that of the target nucleic acid. The probe concentration can be at least 1.1 fold higher than the concentration of the target nucleic acid, for example at least 2, 3, 4, 5, 10, 20, 30, 50, 100 fold or more higher.

The reaction is then denatured to produce a single stranded DNA circle comprising the target nucleic acid and its extension product (See, for example, FIGS. 3-5).

C. Formation and Cleavage of the Cleavage Structure

The covalent circular target nucleic acid is then detected. The covalent circular target nucleic acid can be detected by performing additional annealing and extension reactions. A primer is added under normal hybridization conditions and allowed to hybridize with the P region of the circular target nucleic acid. The primer is extended by a polymerase. The extension strand displaces the 5′ end of the primer (P″) creating a cleavage structure as described herein. The cleavage structure is cleaved with a nuclease to produce nucleic acid fragments which can be detected or measured directly or indirectly by methods known in the art. The nucleic acid fragments can be detected directly, e.g., fluorescent signal from a FRET pair, or indirectly, e.g., secondary cleavage or amplification reaction. (See other related patents, the disclosures of which are incorporated herein by reference for direct detection (U.S. patent application Ser. No. 10/981,942, filed Nov. 5, 2004; U.S. Pat. No. 6,528,254 B1, filed Oct. 29, 1999; U.S. Pat. No. 6,548,250, filed Aug. 30, 2000) and indirect detection (U.S. Pat. No. 6,893,819, filed Nov. 21, 2000; U.S. Application 60/725,916, filed Oct. 11, 2005.) The invention provides for a cleavage structure that can be cleaved by a nuclease, and therefore teaches methods of preparing a cleavage structure. The invention also provides a labeled cleavage structure and methods of preparing a labeled cleavage structure.

In one embodiment, a cleavage structure according to the invention is formed by incubating a) an upstream extendable 3′ end of a first oligonucleotide, e.g., primer (P″ of FIG. 3), b) a second oligonucleotide, e.g, extended circular target nucleic acid (P of FIG. 3) and c) a suitable buffer (for example Sentinel Molecular Beacon PCR core buffer (Catalog #600500) or 10×Pfu buffer available from Stratagene (Catalog #200536), under conditions that allow the primer hybridize to the circular target nucleic acid (for example 95° C. for 2-5 minutes followed by cooling to between approximately 50-60° C.). The optimal temperature will vary depending on the specific probe(s), primers and polymerases.

The 3′ end of the primer is extended by the synthetic activity of a polymerase according to the invention such that the newly synthesized 3′ end of the primer partially displaces its 5′ end. Extension can be performed in the presence of 1× Sentinel Molecular beacon core buffer or 1×Pfu buffer for 15 seconds at 72° C.

In one embodiment the cleaved mononucleotides or small oligonucleotides which are cleaved by the endonuclease activity of the flap-specific nuclease can be detected.

Any of several strategies may be employed to distinguish the uncleaved labeled nucleic acid from the cleaved fragments thereof. In this manner, the present invention permits identification of those samples that contain a target nucleic acid sequence.

The oligonucleotide primer is labeled, as described below, by incorporating moieties detectable by spectroscopic, photochemical, biochemical, immunochemical, enzymatic or chemical means. The method of linking or conjugating the label to the oligonucleotide primer depends, of course, on the type of label(s) used and the position of the label on the primer. Preferably a primer is labeled at the 5′ end although primer labeled at the 3′ end as long as it does not interfere with the 3′ extension of the primer, or labeled throughout the length of the primer are also useful in particular embodiments of the invention.

A variety of labels that would be appropriate for use in the invention, as well as methods for their inclusion in the primer, are known in the art and include, but are not limited to, enzymes (e.g., alkaline phosphatase and horseradish peroxidase) and enzyme substrates, radioactive atoms, fluorescent dyes, chromophores, chemiluminescent labels, electrochemiluminescent labels, such as Origen™ (Igen), that may interact with each other to enhance, alter, or diminish a signal. Of course, if a labeled molecule is used in a PCR based assay carried out using a thermal cycler instrument, the label must be able to survive the temperature cycling required in this automated process.

Fluorophores for use as labels in constructing labeled primer of the invention include rhodamine and derivatives (such as Texas Red), fluorescein and derivatives (such as 5-bromomethyl fluorescein), Lucifer Yellow, IAEDANS, 7-Me₂N-coumarin-4-acetate, 7-OH-4-CH₃-coumarin-3-acetate, 7-NH₂-4-CH₃-coumarin-3-acetate (AMCA), monobromobimane, pyrene trisulfonates, such as Cascade Blue, and monobromorimethyl-ammoniobimane. In general, fluorophores with wide Stokes shifts are preferred, to allow using fluorimeters with filters rather than a monochromometer and to increase the efficiency of detection.

Primers labeled with fluorophores can readily be used in FEN mediated cleavage of a cleavage structure comprising a labeled primer according to the invention. If the label is on the 5′-end of the primer, the FEN generated labeled fragment is separated from the intact, hybridized primer by procedures well known in the art. The fluorescence of the released label is then compared to the label remaining bound to the target. It is not necessary to separate the FEN generated fragment and the primer that remains bound to the target after cleavage in the presence of FEN if the probe is synthesized with a fluorophore, usually at the 5′-end, and a quencher, usually about 20 nucleotides downstream of the dye. Such a dual labeled probe will not fluoresce when intact because the light emitted from the dye is quenched by the quencher. Thus, any fluorescence emitted by an intact primer is considered to be background fluorescence. When a labeled primer is cleaved by a FEN nuclease, dye and quencher are separated and the released fragment will fluoresce. The amount of fluorescence is proportional to the amount of nucleic acid target sequence present in a sample.

In some situations, one can use two interactive labels on a single oligonucleotide with due consideration given for maintaining an appropriate spacing of the labels on the oligonucleotide to permit the separation of the labels during oligonucleotide hydrolysis. Preferred interactive labels useful according to the invention include, but are not limited to rhodamine and derivatives, fluorescein and derivatives, Texas Red, coumarin and derivatives, crystal violet and include, but are not limited to DABCYL, TAMRA and NTB (nitrothiazole blue).

In another embodiment of the invention, detection of the hydrolyzed, labeled primer can be accomplished using, for example, fluorescence polarization, a technique to differentiate between large and small molecules based on molecular tumbling. Large molecules (i.e., intact labeled probe) tumble in solution much more slowly than small molecules. Upon linkage of a fluorescent moiety to the molecule of interest (e.g., the 5′ end of a labeled primer), this fluorescent moiety can be measured (and differentiated) based on molecular tumbling, thus differentiating between intact and digested probe.

Positioning a Reporter Molecule on the Primer

As described above, in one embodiment the invention contemplates the use of a primer that is duel labeled with a fluorophore/quencher pair. When the labeled primer is cleaved (after extension of its 3′ end and displacement of its 5′ end) by a FEN nuclease (or other nuclease as described herein), the fluorphore and quencher are separated and the remaining fragment will fluoresce. It is contemplated that the placement of the fluorophore and quencher on the primer may be specifically selected to discriminate between invasive and non-invasive cleavage of the 5′ flap. It will be appreciated by one of skill in the art that the following disclosure for positioning of reporter molecules, although described in the context of a fluorophore/quencher pair, is not limited to that specific reporter system. The placement of the reporter molecules described below may also be applied to other reporter systems such as, for example, intercalating dyes, wherein the cleavage of the probe at a specific site would result in quenching of the dye signal. The placement scheme described below may also be used in any reporter system comprising interacting reporter molecules, where the interaction of the molecules may be interrupted by cleavage of the non-complementary portion of the downstream probe.

Pfu FEN cleaves the 5′ end of the primer predominantly at a single position relative to the 3′ polymerase extended end of the primer. For example, if the nucleotides of the primer that hybridize to the target are defined to be nucleotides +1 through +X, where +1 is at the end of the 5′ portion of the hybridized region of the primer and +X is at the 3′ end of the hybridized region of the primer (where X equals the number of hybridized residues in the primer). When the 3′ extended end of the primer does not overlap with the 5′ end of the primer's hybridized region, but abuts the 5′ end of the primer such that there is only a nick between the two regions, FEN cleaves between positions +1 and +2 of the 5′ hybridized region of the primer. If the 3′ extended end of the primer has a single 3′ base that overlaps with the 5′ end of the hybridized region of the primer, and hybridizes with the target at that 3′ base position, then cleavage of the primer occurs between positions +2 and +3 of the 5′ hybridized region of the primer. Thus, if the 3′ end of the extended primer overlaps with the 5′ hybridized region of the primer by N nucleotides, and the overlapping region of the 3′ end of the extended primer hybridizes to the target in that region, then cleavage occurs between positions +(N+1) and +(N+2) of the 5′ hybridized region of the primer.

Therefore, where the primer has a pre-formed, non-hybridizing 5′ end (“5′ flap”), cleavage occurs between positions +(N+1) and +(N+2) of the hybridized region of the primer, and release of the 5′ flap from the hybridized region of the primer, provided that the 3′ extended portion of the primer abuts the 5′ hybridized region of the primer, leaving only a nick between the 3′ extended portion and 5′ hybridized protion of the primer, cleavage will occur between positions +1 and +2 of the 5′ hybridized region of the primer and release of the primer's 5′ flap; the 5′ flap will have one nucleotide at its 3′ end derived from the 5′ end of the 5′ hybridized region of the primer. If the 3′ end of the extended primer contains one base at its 3′ end that overlaps with the 5′ hybridized region of the primer, cleavage will occur between positions +2 and +3 of the primer and release of the primer's 5′ flap, now with 2 bases at its 3′ end derived from the first 2 hybridized bases of the primer.

Accordingly, if a reporter group, such as a fluorophore, is attached to base +2 of the 5′ hybridized region of the primer, and a quencher is attached to a base in the 5′ flap, then cleavage downstream of position +2 will leave the quencher and the reporter group still attached to the same nucleic acid molecule. However, if cleavage were to occur upstream of position +2 of the primer (i.e., between positions +1 and +2), then the reporter located on base +2 will become separated from the quencher located on the flap. It also follows that if there is to be cleavage upstream of position +2, the 3′ extended region of the primer cannot have an overlap with the 5′ hybridized region of the primer. Thus, invasive cleavage structures in which the 3′ extended region of the primer has a 3′ portion both complementary to the target and overlapping with the 5′ complementary portion of the downstream oligonucleotide will cause cleavage downstream of position +2 and thus no physical separation of the reporter group located on position +2, and the quencher located in the 5′ flap. Cleavage events resulting from invasive cleavage structures will be undetected, whereas cleavage events resulting from non-invasive cleavage structures will be detectable. Thus, placement of the reporter molecule (such as a fluorophore) at position +2 of the hybridized/complementary portion of the primer permits the discrimination between invasive and non-invasive cleavage. Accordingly, one embodiment of the invention contemplates the placement of the reporter molecule (e.g., fluorophore) at position +2 of the complementary region of the primer. It is also contemplated that the positioning of the fluorophore/quencher pair may be reversed, such that the fluorophore is on the 5′ flap and the quencher is located at position +2 of the complementary region of the primer.

The instant invention is also based, in part, on the discovery that placement of a reporter molecule (such as, but not limited to a fluorophore such as FAM (fluoroscene)) at position +3 or +9 of the primer causes there to be no cleavage between positions +3 and +4 or between positions +9 and +10 of the primer, respectively. Therefore, a primer having a fluorophore (e.g., FAM) at position +2 will not be capable of being cleaved between positions +2 and +3, thus an 3′ extended portion of the primer has a single 3′ overlapping base will not be able to cause cleavage of the primer between positions +2 and +3, whereas a 3′ extended region of the primer with no overlapping region, just a nick, will cause there to be cleavage between positions +1 and +2, which would separate the reporter group from the quencher present on the 5′ flap. Thus, a signal produced by cleavage of a primer with a FAM attached to base +2 of the hybridizing region of the primer will result only from non-invasive cleavage structures, and not from invasive cleavage structures. Moreover, cleavage of a 5′ flap from a primer having a fluophore (e.g., FAM) attached at the +2 position of the complementary region will result in a cleaved flap with one 3′ base, or three 3′ bases from the 5′ end of the complementary region attached to the 3′ end of the flap. Such a flap cannot have exactly 2 additional bases attached to its 3′ end because the cleavage structure that would result in two 3′ bases being left attached to the 5′ flap would not be cleaved because of blockage of FEN by the fluorophore on the +2 base of the probe.

Accordingly, the invention contemplates the use of downstream probes having a fluorophore group (such as, but not limited to a FAM group) on base +2 and a quencher upstream (5′ prime) of position +1 to generate signals only from non-invasive cleavage structures, and not from invasive cleavage structures.

D. Detection of Released Fragments

Detection or verification of the fragments may be accomplished by a variety of methods well known in the art and may be dependent on the characteristics of the labeled moiety or moieties comprising a cleavage structure.

The released fragments can be detected directly, e.g., fluorescent signal from a FRET pair, or indirectly, e.g., secondary cleavage or amplification reaction. (See other related patents, the disclosures of which are incorporated herein by reference for direct detection (U.S. patent application Ser. No. 10/981,942, filed Nov. 5, 2004; U.S. Pat. No. 6,528,254 B1, filed Oct. 29, 1999; U.S. Pat. No. 6,548,250, filed Aug. 30, 2000) and indirect detection (U.S. Pat. No. 6,893,819, filed Nov. 21, 2000; U.S. Application 60/725,916, filed Oct. 11, 2005.)

In one embodiment of the invention, the reaction products, including the released labeled fragments, are subjected to size analysis. Methods for determining the size of a labeled fragment are known in the art and include, for example, gel electrophoresis, sedimentation in gradients, gel exclusion chromatography, mass spectroscopy, and homochromatography.

During or after amplification, separation of the released labeled fragments from, for example, a PCR mixture can be accomplished by, for example, contacting the PCR with a solid phase extractant (SPE). For example, materials having an ability to bind nucleic acids on the basis of size, charge, or interaction with the nucleic acid bases can be added to the PCR mixture, under conditions where labeled, uncleaved nucleic acids are bound and short, labeled fragments are not. Such SPE materials include ion exchange resins or beads, such as the commercially available binding particles Nensorb (DuPont Chemical Co.), Nucleogen (The Nest Group), PEI, BakerBond™ PEI, Amicon PAE 1,000, Selectacel™ PEI, Boronate SPE with a 3′-ribose probe, SPE containing sequences complementary to the 3′-end of the probe, and hydroxylapatite. In a specific embodiment, if a dual labeled oligonucleotide comprising a 3′ biotin label separated from a 5′ label by a nuclease susceptible cleavage site is employed as the signal means, the reaction mixture, for example a PCR amplified mixture can be contacted with materials containing a specific binding partner such as avidin or streptavidin, or an antibody or monoclonal antibody to biotin. Such materials can include beads and particles coated with specific binding partners and can also include magnetic particles.

Following the step in which a reaction mixture, for example a PCR mixture has been contacted with an SPE, the SPE material can be removed by filtration, sedimentation, or magnetic attraction, leaving the labeled fragments free of uncleaved labeled oligonucleotides and available for detection.

### EXAMPLES

The invention is illustrated by the following nonlimiting examples wherein the following materials and methods are employed. The entire disclosure of each of the literature references cited hereinafter are incorporated by reference herein.

### Example 1

A target nucleic acid sequence can be detected and/or measured by the following method. A labeled cleavage structure is formed prior to the addition of a FEN nuclease by heating at 95° C. for 5 minutes and then cooling to approximately 50-60° C. (a) a sample containing a target nucleic acid sequence with (b) an upstream oligonucleotide that specifically hybridizes to the target nucleic acid sequence, and (c) a downstream, 5′ end labeled oligonucleotide that specifically hybridizes to a region of the target nucleic acid sequence that is downstream of the hybridizing region of the upstream oligonucleotide. A polymerase that lacks a 5′ to 3′ exonuclease activity but that possesses a 3′ to 5′ DNA synthetic activity, such as the enzyme a) Yaq exo−, (prepared by mutagenesis using the Stratagene QuikChange Site-Directed Mutagenesis kit, catalog number #200518, to modify Taq polymerase (Tabor and Richardson, 1985, Proc. Natl. Acad. Sci. USA, 82:1074)), a mutant form of Taq polymerase that lacks 5′ to 3′ exonuclease activity, b) Pfu, or c) a mutant form of Pfu polymerase that lacks 3′ to 5′ exonuclease activity (exo− Pfu) is added and incubated under conditions that permit the polymerase to extend the upstream oligonucleotide such that it partially displaces the 5′ end of downstream oligonucleotide (for example 72° C. in 1×Pfu buffer (Stratagene) for 5 minutes to 1 hour. The downstream oligonucleotide has a 5′ flap that is cleaved upon the addition of a FEN nuclease.

A mutant form of Taq polymerase that lacks a 5′ to 3′ exonuclease activity but that possesses a 3′ to 5′ DNA synthetic activity comprises the following mutation: D144S/F667Y Taq wherein D144S eliminates 5′ to 3′ exonuclease activity and F667Y improves ddNTP incorporation.

Exo− mutants of PolI polymerase can be prepared according to the method of Xu et al., 1997, J. Mol. Biol., 268: 284.

A labeled cleavage structure according to the invention is cleaved with a preparation of PfuFEN-1 (i.e. cloned Pyrococcus furiosus FEN-1). Cleavage is carried out by adding 2 μl of PfuFEN-1 to a 7 μl reaction mixture containing the following:

3 μl cleavage structure (10 ng-10 μg)

0.7 μl 10×FEN nuclease buffer (10×FEN nuclease buffer contains 500 mM Tris-HCl pH 8.0, 100 mM MgCl₂)

2.00 μl PfuFEN-1 enzyme or H₂O

1.3 μl H₂O

7.00 μl total volume

Samples are incubated for one hour at 50° C. in a thermal cycler. Following the addition of 2 μl of Sequencing Stop dye solution (included in the Stratagene Cyclist DNA sequencing kit, catalog #200326, and described in example 3), samples are heated at 99° C. for five minutes. Samples are loaded on an eleven inch long, hand-poured, 20% acrylamide/bis acrylamide, 7M urea gel. The gel is run at 20 watts until the bromophenol blue has migrated approximately ⅔ the total distance. The gel is removed from the glass plates and soaked for 10 minutes in fix solution (15% methanol, 5% acetic acid) and then for 10 minutes in water. The gel is placed on Whatmann 3 mm paper, covered with plastic wrap and dried for 2 hours in a heated vacuum gel dryer (˜800 C). The gel is exposed overnight to X-ray film to detect the presence of a signal that is indicative of the presence of a target nucleic acid sequence.

### Example 2

Description of Probes:

FIG. 3 is a schematic depicting an embodiment of the circular cleavage reaction. In this example, the reaction can be performed using the reaction conditions (e.g, buffers, incubation times and temperatures) described in U.S. Pat. No. 6,528,254 B1, filed Oct. 29, 1999 and U.S. Pat. No. 6,548,250, filed Aug. 30, 2000 both of which are herein incorporated by reference in their entireties.

The method uses a target nucleic acid (Y1, Y2), a probe (Y1′, Y2′ P ′) and a primer P″. Generally each region is about 15-20 nt's long.

The target can be any oligonucleotide that has a 3′ OH. For example, the target can be generated during an earlier amplification reaction or cleavage reaction. Generally the target is the 3′ region of an amplified nucleic acid and has a region 5′ of Y1 that forms a flap when hybridized to the probe. The probe preferably has a blocked 3′ end. The Y1′ and Y2′ regions of the probe are complementary to the Y1 and Y2 regions of the target. In a preferred embodiment the reaction mixture includes a polymerase, ligase and nuclease, e.g., FEN. Such reaction mixtures are described in U.S. application Ser. No. 10/198,449, filed Jul. 18, 2002, which is herein incorporated by reference in its entirety.

Description of the Method:

Anneal/Extension: The Y1 and Y2 regions of the target anneal to the Y1′ and Y2′ regions of the probe forming a duplex. The 5′ and 3′ ends of the probe are bridged by the target. The 3′ region of the target is then extended by a polymerase producing an extension product that is complementary to the probe.

Cleavage: In some embodiments, if there is a 5′ flap on the target nucleic acid, the extension product forms a cleavage structure with the 5′ flap of the target which can then be cleaved by the cleavage agent. A ligase can then join the 5′ end of the cleaved target with the extension product producing a double-stranded (ds) circular nucleic acid. Alternatively, if there is no 5′ flap on the target, the 5′ end of the target is simply ligated to the extension product (provided 5′ end of target is phosphorylated) and no cleavage reaction takes place during this step of the reaction. After ligation of the extended product and the target, the reaction is denatured generating a single-stranded (ss) circular DNA molecule having Y1, Y2 and P regions.

Anneal/Extension: During the next annealing step the primer (P″) anneals to the ss circular DNA molecule. The primer is extended through both the Y1 and Y2 regions of the circular DNA to the 5′ end of itself.

Cleavage: The polymerase displaces the 5′ region of the primer producing a cleavage structure. Alternatively, the polymerase extends the primer up to a preformed 5′ flap, thus creating a cleavage structure. The cleavage structure is cleaved by the cleavage agent generating a free 5′ flap. The cleavage reaction may directly produce a detectable signal (see U.S. Pat. No. 6,528,254 B1 (filed Oct. 29, 1999) and U.S. Pat. No. 6,548,250 (filed Aug. 30, 2000), and U.S. Ser. No. 10/981,942 (filed Nov. 5, 2004), the disclosures of all of which are incorporated herein by reference), or the flap may be detected by an indirect detection method (see U.S. Provisional Application 60/725,916 (filed Oct. 11, 2005), the disclosure of which is incorporated herein by reference and see also U.S. Pat. No. 6,893,819 (filed Nov. 21, 2000), the disclosure of which is incorporated herein by reference.

### Example 3

Description of Probes:

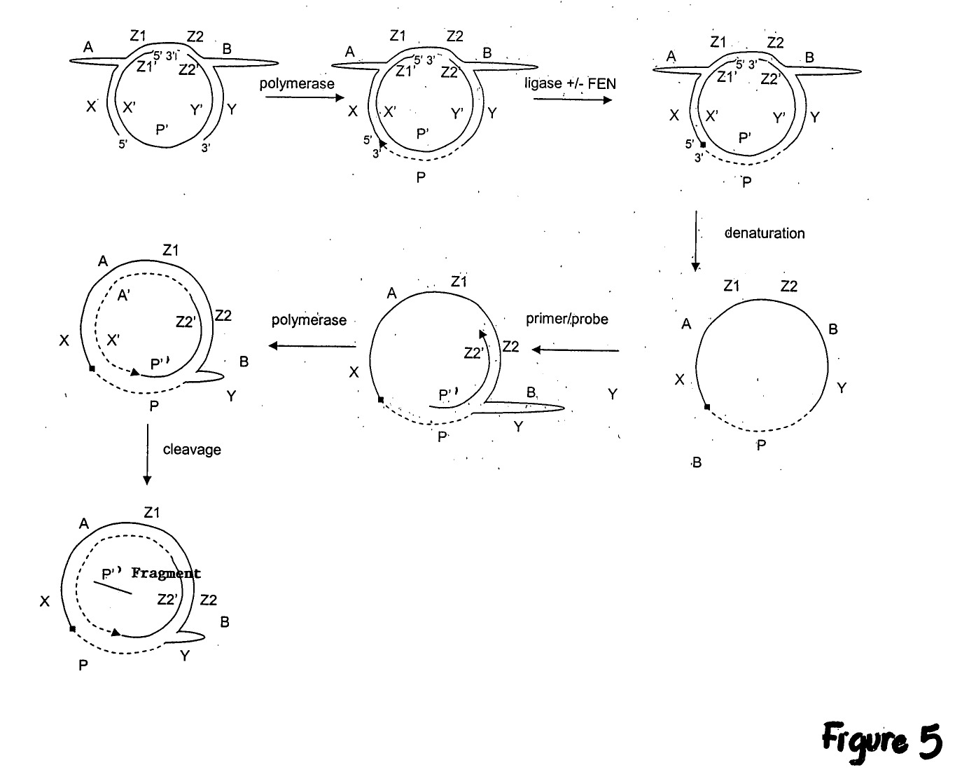

FIG. 4 is a schematic depicting an embodiment of the circular cleavage reaction, which is a modification of the cleavage reaction described for Example 2 (FIG. 3). In this embodiment, the target is synthesized in a nucleic acid amplification reaction. The target includes a B region, and the probe contains a complementary B′ region. The primer has P″ and Y2′ regions. The B region is the binding portion for a reverse PCR primer that is used to generate the target. The P″ and Y2′ regions of the primer can be 10-20 nucleotides (nts) and should be designed so that neither can anneal to the ss circular DNA molecule independently. If B′ binds to the ss circular DNA molecule P″Y2′ can not bind.

Description of the Method:

The method of this example is a modification of the method of Example 2. In this embodiment the primer (P″Y2′) anneals to the ss circular DNA molecule and loops out the B region (reverse primer binding region). Otherwise for the purposes of this example the method is similar to that of Example 2.

### Example 4

Description of Probes:

FIG. 5 is a schematic depicting an embodiment of the circular cleavage reaction, which is a modification of the cleavage reaction described for Examples 2 and 3 (FIG. 3 and FIG. 4). In this example, the target includes X, A, Z1, Z2, B, and Y regions. The target is created by a PCR reaction utilizing PCR primers that are complementary to the A and B regions of the target. The PCR primers also include X and Y regions which are incorporated into the amplicon and are now regions within the target.

The primer has P″ and Z2′ regions.

The probe includes P′, Y′, X1, Z2′ and Z1 regions. The X, A, B, Y and (Z1/Z2) are each about 20 nts thus forming a target of about 100 nts. The probe is about 60 nts long.

Description of the Method:

The method of this example is a modification of the methods of Examples 2 and 3. In this example, the primer (P″Z2′) anneals to the ss circular DNA molecule and loops out the BY region (reverse primer binding region). Also, when the target binds to the probe the A and B portions are looped out. Otherwise, for the purposes of this example, the method is similar to those of Examples 2 and 3.

### Example 5

Example 5 describes the detection of target nucleic acid with specific 5′ and 3′-ends by generation of a circular DNA product using a probe with 5′ and 3′ ends that are complementary to the 5′ and 3′ ends of the target.

Target Molecule Sequence:

5′ P-AGTCTAAGGCTGGAATAACTAACCTGCTCACGCGCATCAC 3′

The target molecule was phosphorylated at the 5′-end to allow closure of the circular DNA product. Bold and italic nucleotides are complementary to the corresponding highlighted sequences of the probe.

Probe sequence
5′-AGTTATTCCAGCCTTAGACTCCCCTTATGGGAACGAGCAGACCG [symbol] [symbol]
CGTTGAGCCATCGCTGTATCGTGATGCGCGTGA
GCAGGT-T 3′

The T-T sequence at the 3′ prime end indicates a 3′-3′ linkage between the last and the second to last nucleotide introduced to prevent extension of the 3′-end of the probe by the polymerase when annealed to the target molecule. The underlined nucleotides indicate a 2′-methoxy modification of the backbone that prevent Pfu-polymerase-based DNA synthesis past the modification, thus preventing strand-displacement of the annealed 3′-end of the target to the probe. Sequence in bold italics indicate PCR-primer annealing sites used in the subsequent PCR amplification.

PCR primer
GTCTTGCGGAGGTCATCACT
and
CGTTGAGCCATCGCTGTATC

Experimental Conditions:

The target-detection was carried out in a 50 μl reaction volume containing concentrations of target from 0.05-5 fmoles, 50-fmoles probe, 1×-cloned Pfu buffer (Stratagene), 2.5 U Pfu-TURBO (Stratagene), 30 U Tsc-ligase (Stratagene), 10 mmoles of each dNTP and 30 fmoles of each PCR primer.

Cycling conditions: 1 cycle of 95 oC for 1 minute and 60 oC for 1 minute followed by 25 cycles of 95° C. for 30 seconds, 55° C. for 30 seconds and 72° C. for 30 seconds. PCR products were analyzed by separation on a 3.5% NuSieve gel and visualization of the products by ethidium bromide staining The target nucleic acid was detected at a concentration as low as 0.05 fmoles under the selected conditions.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

What is claimed is:

1. A method of generating a signal indicative of the presence of a target nucleic acid in a sample, wherein the signal generated indicates the presence of an extension sequence resulting from extension of the target nucleic acid, the method comprising:
a) extending the target nucleic acid to add an additional region;
b) circularizing the extended target nucleic acid;
c) forming a cleavage structure by incubating the sample with a nucleic acid polymerase, the cleavage structure comprising duplex and single-stranded nucleic acid, wherein the single-stranded nucleic acid comprises a 5′ flap;
d) cleaving the cleavage structure with a nuclease to release a nucleic acid fragment from the cleavage structure; and
e) detecting and/or measuring the release of the nucleic acid fragment as an indication of the presence of the target nucleic acid.

2. A method of generating a signal indicative of the presence of a target nucleic acid in a sample, wherein the signal generated indicates the presence of an extension sequence resulting from extension of the target nucleic acid, the method comprising:
a) extending the target nucleic acid to add an additional region;
b) circularizing the extended target nucleic acid;
c) forming a cleavage structure by incubating the sample with a primer which hybridizes to the circularized and extended target nucleic acid and extending the 3′ end of the primer with a nucleic acid polymerase and displacing the 5′ end of the primer;
d) cleaving the cleavage structure with a nuclease to release a nucleic acid fragment from the cleavage structure; and
e) detecting and/or measuring the release of the nucleic acid fragment as an indication of the presence of the target nucleic acid.

3. The method of claim 1, wherein the nucleic acid polymerase substantially lacks 5′ to 3′ exonuclease activity.

4. The method of claim 1, wherein the nucleic acid polymerase is a DNA polymerase.

5. The method of claim 1, wherein the nucleic acid polymerase is thermostable.

6. The method of claim 1, wherein the nucleic acid polymerase is selected from the group consisting of 5′ to 3′ exonuclease deficient Taq polymerase and Pfu polymerase.

7. The method of claim 1, wherein a cleavage structure is formed comprising at least one labeled moiety capable of providing a signal.

8. The method of claim 1, wherein the nuclease comprises a FEN nuclease.

9. The method of claim 1, wherein the FEN nuclease is a flap-specific nuclease.

10. The method of claim 1, wherein the FEN nuclease is thermostable.

11. The method of claim 10, wherein the FEN nuclease is selected from the group consisting of FEN nuclease enzyme derived from Archaeglobus fulgidus, Methanococcus jannaschii, Pyrococcus furiosus.

12. The method of claim 10, wherein the cleavage structure formed comprises a pair of interactive signal generating labeled moieties effectively positioned to quench the generation of a detectable signal, the labeled moieties being separated by a site susceptible to FEN nuclease cleavage, thereby allowing the nuclease activity of the FEN nuclease to separate the first interactive signal generating labeled moiety from the second interactive signal generating labeled moiety by cleaving at the site susceptible to FEN nuclease, thereby generating a detectable signal.

13. The method of claim 12, wherein the pair of interactive signal generating moieties comprises a quencher moiety and a fluorescent moiety.

14. The method of claim 1, wherein the cleavage structure comprises at least one oligonucleotide primer.

15. The method of claim 1, further comprising quantifying the released nucleic acid fragment to calculate the amount of target nucleic acid in the sample.

16. A method for forming a cleavage structure in a sample, cleaving the cleavage structure to produce a cleavage product that is indicative of the presence of the target nucleic acid, and transcribing a nucleic acid complementary to the target nucleic acid, wherein the method comprises:
a) providing a sample comprising a target nucleic acid and a nucleic acid probe, wherein:
i) the target nucleic acid comprises:
A) a 3′ region; and
B) an upstream region; and
ii) the nucleic acid probe comprises:
A) a 5′ region complementary to the upstream region on the target nucleic acid;
B) a 3′ region complementary to the 3′ region on the target nucleic acid; and
C) an additional region;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region downstream of the 3′ region and complementary to the additional region of the nucleic acid probe, followed by circularizing the extended target nucleic acid;
d) removing the nucleic acid probe from the extended target nucleic acid;
e) annealing a primer to the extended target nucleic acid, wherein the primer has a sequence that is complementary to at least a portion of the primer binding region on the extended target nucleic acid and primes the synthesis of a complementary nucleic acid strand;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

17. The method of claim 16, wherein the 3′ end of the target nucleic acid in step a comprises a 3′ hydroxyl group.

18. The method of claim 16, wherein the nucleic acid probe has a blocked 3′ end.

19. The method of claim 16, wherein the blocked 3′ end comprises a base that is non-complementary to the target nucleic acid or a modification that inhibits addition of a nucleotide triphosphate under conditions which permit the nucleic acid synthesis or extension.

20. The method of claim 19, wherein one or both of the blocked 3′ end comprises:
a) a dideoxynucleotide;
b) a nucleotide wherein the 3′ hydroxyl has been replaced with a phosphate group; or
c) a nucleotide with a reporter moiety attached to the 3′ carbon or to the 3′ oxygen.

21. The method of claim 16, wherein the circularization of the extended target nucleic acid comprises:
i) self-ligation of the extended target nucleic acid; or
ii) cleavage of a non-complementary 5′ portion of the extended target and self-ligation.

22. A method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
i) the target nucleic acid comprises:
A) a 3′ region (Y2); and
B) a region (Y1) upstream to Y2; and
ii) the nucleic acid probe comprises:
A) a 5′ region (Y1′) complementary to Y1;
B) a 3′ region (Y2′) complementary to Y2; and
C) an additional binding region (P′) between Y1′ and Y2′;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region (P), wherein P is downstream of Y2 and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid and primes the synthesis of a complementary nucleic acid strand;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

23. A method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
i) the target nucleic acid comprises:
A) a 3′ region (B);
B) a region (Y2) upstream to B; and
C) a region (Y1) upstream to Y2; and
ii) the nucleic acid probe comprises:
A) a 5′ region (Y1′) complementary to Y1;
B) a 3′ region (Y2′) complementary to Y2;
C) a region (B′) upstream to Y2′; and
D) an additional region (P′) between Y1′ and B′;
b) annealing the nucleic acid probe to the target nucleic acid and priming synthesis of an extended target nucleic acid;
c) incubating the sample with a nucleic acid polymerase and extending the 3′ end of the target nucleic acid to generate an extended target nucleic acid having a primer binding region (P), wherein P is downstream of B and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid upstream of a sequence complementary to at least a portion of region Y2 and primes the synthesis of a complementary nucleic acid strand while looping out region B of the extended target nucleic acid;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

24. A method for forming a cleavage structure in a sample, cleaving the cleavage structure to produce a cleavage product that is indicative of the presence of the target nucleic acid in the sample, and transcribing a nucleic acid complementary to the target nucleic acid, wherein the method comprises:
a) providing a sample comprising a target nucleic acid and a nucleic acid probe, wherein:
i) the target nucleic acid comprises:
A) a 3′ region (Y);
B) a region (X) upstream of Y; and
C) a series of intervening regions (A, Z1, Z2, B) between X and Y in the order of X-A-Z1-Z2-B-Y;
ii) the nucleic acid probe comprises:
A) a 5′ region (Z1′) complementary to Z1;
B) a 3′ region (Z2′) complementary to Z2;
C) a region (X′) complementary to X;
D) a region (Y′) complementary to Y; and
E) an additional region (P′) between X1′ and Y1′;
b) annealing the nucleic acid probe to the target nucleic acid and priming synthesis of an extended target nucleic acid while looping out one or more portions of regions A and B of the target sequence;
c) incubating the sample with a nucleic acid polymerase and extending the 3′ end of the target nucleic acid to generate an extended target nucleic acid having a primer binding region (P), wherein P is downstream of Y and complementary to P′ and circularizing the extended target nucleic acid to circularize the extended target nucleic acid;
d) removing the nucleic acid probe from the extended target nucleic acid;
e) annealing a primer to the extended target nucleic acid, wherein the primer comprises a sequence complementary to at least a portion of region P on the extended target nucleic acid upstream of a sequence complementary to at least a portion of region Z2 and primes the synthesis of a complementary nucleic acid strand while looping out one or more portions of regions Y and B of the extended target nucleic acid;
f) extending the primer of step (e) wherein said nucleic acid polymerase synthesizes a primer extension product, and wherein the primer extension product partially displaces its 5′ end to form a cleavage structure;
g) cleaving the cleavage structure with a nuclease to release a nucleic acid fragment from the cleavage structure; and
h) detecting and/or measuring the release of the nucleic acid fragment as an indication of the presence of the target nucleic acid.

25. A kit for generating a signal indicative of the presence of a target nucleic acid sequence of a target nucleic acid in a sample, comprising a nucleic acid polymerase, a FEN nuclease, a suitable buffer, a nucleic acid probe, wherein:
i) the target nucleic acid comprises:
A) a 3′ region; and
B) an upstream region; and
ii) the nucleic acid probe comprises:
A) a 5′ region complementary to the upstream region on the target nucleic acid;
B) a 3′ region complementary to the 3′ region on the target nucleic acid; and
C) an additional region.

26. The kit of claim 25, wherein the nucleic acid probe has a blocked 3′ end.

27. The kit of claim 25 wherein the nucleic acid polymerase substantially lacks 5′ to 3′ exonuclease activity.

28. The kit of claim 25 wherein the nucleic acid polymerase is thermostable.

29. The kit of claim 25 wherein the FEN nuclease is thermostable.

30. The kit of claim 25 further comprising a primer, wherein the primer has a sequence that comprises at least a portion of the sequence of the additional region of the nucleic acid probe.

31. The kit of claim 30, wherein the primer is labeled.

32. A method of detecting a target nucleic acid comprising:
a) annealing the target nucleic acid to a nucleic acid probe so as to circularize said probe;
b) extending the target nucleic acid to form a circularized target-probe duplex; and
c) detecting and/or measuring the circularized target-probe duplex.

33. The method of claim 32, wherein said target nucleic acid comprises a 3′ region and an upstream region.

34. The method of claim 33, wherein said nucleic acid probe comprises a 5′ region complementary to the upstream region on said target nucleic acid and a 3′ region complementary to the 3′ region on said target nucleic acid.

35. A method of detecting a target nucleic acid comprising:
a) annealing an upstream region of said target nucleic acid to a 5′ region of a probe and annealing a 3′ region of said target nucleic acid to a 3′ region of said probe;
b) extending the target nucleic acid to form a region complementary to said probe; and
c) detecting and/or measuring the reaction extended target.
